SEQUENCE LISTING (23 sequences)

SEQ ID NO: 1 - protein, 655 aa, Mus sp.
MVQSDTSKSPPVAAVAQESQMELLESAAPAGALGAQSYGKGARRKNRFKGSDGSTSSDTTSNSFVRQGSADSYTSRPSDSDVSLEEDREAVRREAERQAQAQLEKAKTKPVAFAVRTNVRYSAAQEDDVPVPGMAISFEAKDFLHVKEKFNNDWWIGRLVKEGCEIGFIPSPVKLENMRLQHEQRAKQGKFYSSKSGGNSSSSLGDIVPSSRKSTPPSSAIDIDATGLDAEENDIPANHRSPKPSANSVTSPHSKEKRMPFFKKTEHTPPYDVVPSMRPVVLVGPSLKGYEVTDMMQKALFDFLKHRFEGRISITRVTADISLAKRSVLNNPSKHAIIERSNTRSSLAEVQSEIERIFELARTLQLVVLDADTINHPAQLSKTSLAPIIVYVKISSPKVLQRLIKSRGKSQAKHLNVQMVAADKLAQCPPQESFDVILDENQLEDACEHLADYLEAYWKATHPPSGNLPNPLLSRTLASSTLPLSPTLASNSQGSQGDQRPDRSAPRSASQAEEEPCLEPVKKSQHRSSSATHQNHRSGTGRGLSRQETFDSETQESRDSAYVEPKEDYSHEHVDRYVPHREHNHREETHSSNGHRHRESRHRSRDMGRDQDHNECIKQRSRHKSKDRYCDKEGEVISKRRNEAGEWNRDVYIRQ

SEQ ID NO: 2 - protein, 660 aa, Homo sapiens
MVQRDMSKSPPTAAAAVAQEIQMELLENVAPAGALGAAAQSYGKGARRKNRFKGSDGSTSSDTTSNSFVRQGSADSYTSRPSDSDVSLEEDREAVRREAERQAQAQLEKAKTKPVAFAVRTNVSYSAAHEDDVPVPGMAISFEAKDFLHVKEKFNNDWWIGRLVKEGCEIGFIPSPVKLENMRLQHEQRAKQGKFYSSKSGGNSSSSLGDIVPSSRKSTPPSSAIDIDATGLDAEENDIPANHRSPKPSANSVTSPHSKEKRMPFFKKTEHTPPYDVVPSMRPVVLVGPSLKGYEVTDMMQKALFDFLKHRFEGRISITRVTADISLAKRSVLNNPSKHAIIERSNTRSSLAEVQSEIERIFELARTLQLVVLDADTINHPAQLSKTSLAPIIVYVKISSPKVLQRLIKSRGKSQAKHLNVQMVAADKLAQCPPELFDVILDENQLEDACEHLADYLEAYWKATHPPSSSLPNPLLSRTLATSSLPLSPTLASNSQGSQGDQRTDRSAPIRSASQAEEEPSVEPVKKSQHRSSSSAPHHNHRSGTSRGLSRQETFDSETQESRDSAYVEPKEDYSHDHVDHYASHRDHNHRDETHGSSDHRHRESRHRSRDVDREQDHNECNKQRSRHKSKDRYCEKDGEVISKKRNEAGEWNRDVYIRQ

SEQ ID NO: 3 - protein, 178 aa, Mus sp.
ASSTLPLSPTLASNSQGSQGDQRPDRSAPRSASQAEEEPCLEPVKKSQHRSSSATHQNHRSGTGRGLSRQETFDSETQESRDSAYVEPKEDYSHEHVDRYVPHREHNHREETHSSNGHRHRESRHRSRDMGRDQDHNECIKQRSRHKSKDRYCDKEGEVISKRRNEAGEWNRDVYIRQ

SEQ ID NO: 4 - protein, 180 aa, Homo sapiens
ATSSLPLSPTLASNSQGSQGDQRTDRSAPIRSASQAEEEPSVEPVKKSQHRSSSSAPHHNHRSGTSRGLSRQETFDSETQESRDSAYVEPKEDYSHDHVDHYASHRDHNHRDETHGSSDHRHRESRHRSRDVDREQDHNECNKQRSRHKSKDRYCEKDGEVISKKRNEAGEWNRDVYIRQ

SEQ ID NO: 5 - protein, 26 aa, Mus sp.
KGYLDWITQAEDIDPENEDEGMDEDK

SEQ ID NO: 6 - protein, 26 aa, Rattus sp.
KGYLDWITQAEDIDPENEDEGMDEDK

SEQ ID NO: 7 - protein, 26 aa, Homo sapiens
KGYLDWITQAEDIDPENEDEGMDEEK

SEQ ID NO: 8 - protein, 14 aa, Mus sp.
KSITADGESPPTTK

SEQ ID NO: 9 - protein, 14 aa, Rattus sp.
KSITADGESPPTTK

SEQ ID NO: 10 - protein, 22 aa, Mus sp.
HSNPDTAGEEDEEEPEMPVGPR

SEQ ID NO: 11 - protein, 22 aa, Rattus sp.
HSNPDTAGEEDEEEPEMPVGPR

SEQ ID NO: 12 - protein, 25 aa, Homo sapiens
KSPYPNPETTGEEDEEEPEMPVGPR

SEQ ID NO: 13 - protein, 14 aa, Mus sp.
KTGNNQADTESPSH

SEQ ID NO: 14 - protein, 14 aa, Rattus sp.
KTGNNQADTESPSH

SEQ ID NO: 15 - protein, 26 aa, Mus sp.
RMSEEAEYSEPSLLSTDMFSYQEDEH

SEQ ID NO: 16 - protein, 25 aa, Homo sapiens
HDTEACSEPSLLSTEMLSYQDDENR

SEQ ID NO: 17 - protein, 13 aa, Homo sapiens
RGAPSEEELQDSR

SEQ ID NO: 18 - protein, 26 aa, Mus sp.
KGYLDWITQAEDIDPENEDEGMDEDK

SEQ ID NO: 19 - protein, 26 aa, Homo sapiens
KGYLDWITQAEDIDPENEDEGMDEEK

SEQ ID NO: 20 - protein, 15 aa, Artificial
  synthetically constructed H sequence (mouse and human)
GEEDEEEPEMPVGPR

SEQ ID NO: 21 - protein, 2139 aa, Mus sp.
MVNENTRMYVPEENHQGSNYGSPRPAHANMNANAAAGLAPEHIPTPGAALSWQAAIDAARQAKLMGSAGNATISTVSSTQRKRQQYGKPKKQGGTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIIDVPAEEDPSPCALETGHGRQCQNGTVCKPGWDGPKHGITNFDNFAFAMLTVFQCITMEGWTDVLYWMQDAMGYELPWVYFVSLVIFGSFFVLNLVLGVLSGEFSKEREKAKARGDFQKLREKQQLEEDLKGYLDWITQAEDIDPENEDEGMDEDKPRNMSMPTSETESVNTENVAGGDIEGENCGARLAHRISKSKFSRYWRRWNRFCRRKCRAAVKSNVFYWLVIFLVFLNTLTIASEHYNQPHWLTEVQDTANKALLALFTAEMLLKMYSLGLQAYFVSLFNRFDCFIVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQEVMEKPAVEESKEEKIELKSITADGESPPTTKINMDDLQPSENEDKSPHSNPDTAGEEDEEEPEMPVGPRPRPLSELHLKEKAVPMPEASAFFIFSPNNRFRLQCHRIVNDTIFTNLILFFILLSSISLAAEDPVQHTSFRNHILGNADYVFTSIFTLEIILKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKHYFCDAWNTFDALIVVGSIVDIAITEVHPAEHTQCSPSMSAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVTYYQSDSRGNFPQTFATQRPLHINKTGNNQADTESPSHEKLVDSTFTPSSYSSTGSNANINNANNTALGRFPHPAGYSSTVSTVEGHGPPLSPAVRVQEAAWKLSSKRCHSRESQGATVNQEIFPDETRSVRMSEEAEYCSEPSLLSTDMFSYQEDEHRQLTCPEEDKREIQPSPKRSFLRSASLGRRASFHLECLKRQKDQGGDISQKTALPLHLVHHQALAVAGLSPLLQRSHSPTTFPRPCPTPPVTPGSRGRPLRPIPTLRLEGAESSEKLNSSFPSIHCSSWSEETTACSGSSSMARRARPVSLTVPSQAGAPGRQFHGSASSLVEAVLISEGLGQFAQDPKFIEVTTQELADACDMTIEEMENAADNILSGGAQQSPNGTLLPFVNCRDPGQDRAVVPEDESCAYALGRGRSEEALADSRSYVSNL

SEQ ID NO: 22 - protein, 2135 aa, Homo sapiens [sequence omitted]

SEQ ID NO: 23 - protein, 5 aa, Artificial
  synthetically constructed Akt consensus sequence
RTDRS

(19) United States
(12) Patent Application Publication
(10) Pub. No.: US 2012/0070451 A1
(43) Pub. Date: Mar. 22, 2012

(54) METHODS AND COMPOSITIONS FOR MODULATING CARDIAC CONTRACTILITY

(75) Inventors: Gianluigi Condorelli, Rome (IT); Deniele Catalucci, Milan (IT)

(73) Assignee: Consiglio Nazionale Delle Ricerche, Rome (IT)

(21) Appl. No.: 13/257,590

(22) PCT Filed: Mar. 19, 2010
(86) PCT No.: PCT/EP2010/002170

§ 371 Date: Dec. 2, 2011

(30) Foreign Application Priority Data
Mar. 19, 2009 (GB) 0904743.2

(51) Int. Cl. A61K 38/17; A61K 31/7105; A61P 9/04; A61P 9/00; C07K 14/435; C07H 21/02; A61K 31/711; A61K 31/713

(52) U.S. Cl. 424/178.1; 514/16.4; 514/44 R; 514/44 A; 530/350; 536/23.5

(57) ABSTRACT

Provide is a Caᵥβ2 peptide or variant thereof, or synthetic molecules, or polynucleotides encoding said peptide or variant, for use in the modulation of cardiac inotropism. Also provided are compositions and methods of treatment comprising said Caᵥβ2 peptide, polynucleotide or variants thereof.

### FIELD OF THE INVENTION

The present invention relates to a Caᵥβ2 peptide or functional variant thereof, or polynucleotides encoding said peptide or variant, for use in the modulation of cardiac inotropism or cardiac contractility.

### BACKGROUND TO THE INVENTION

The insulin-like growth factor-1 (IGF-1)/phosphatidyl-inositol 3-kinase (PI3K)/Akt pathway plays a crucial role in a broad range of biological processes involved in the modulation of local responses as well as processes implicated in metabolism, cell proliferation, transcription, translation, apoptosis, and growth. In the heart, the IGF-1/PI3K/Akt pathway is involved in the regulation of contractile function and impairment of this signaling pathway is considered an important determinant of cardiac function (Catalucci and Condorelli, 2006; Ceci et al., 2004; Condorelli et al., 2002; McMullen et al., 2004; McMullen et al., 2003; Sun et al., 2006).

The Akt (also called PKB) family of serine/threonine kinases consists of 3 isoforms (Akt-1, -2, and -3) that are activated by IGF-1 and insulin through PI3K, a member of the lipid kinase family involved in the phosphorylation of membrane phosphoinositides (Ceci et al., 2004). The product of PI3K binds to the pleckstrin domain of Akt and induces its translocation from the cytosol to the plasma membrane where Akt becomes accessible for phosphorylation by phosphoinositide-dependent kinase-1 (PDK1), resulting in its activation (Bayascas et al., 2008; Ceci et al., 2004). The Ca²⁺ current (I_{Ca,L}) in both cardiomyocytes and neuronal cells has been shown to be increased by Akt activation (Blair et al., 1999; Catalucci and Condorelli, 2006; Sun et al., 2006; Viard et al., 2004) and decreased by Akt inhibition (Catalucci and Condorelli, 2006; Sun et al., 2006; Viard et al., 2004), suggesting a pivotal role of Akt in regulating L-type Ca²⁺ channel complex (LTCC) function.

In cardiomyocytes, the LTCC is composed of different subunits: the pore-forming subunit Caᵥα1, and the accessory β, and α2δ subunits (Bourinet et al., 2004; Catterall, 2000). The opening of the LTCC is primarily regulated by the membrane potential and by other factors, including a variety of hormones, protein kinases, phosphatases, and accessory proteins (Bodi et al., 2005). In healthy cardiomyocytes, electrical excitation starting during the upstroke of the action potential leads to cytosolic Ca²⁺ influx through opening of the LTCC (Bers and Perez-Reyes, 1999; Richard et al., 2006). This triggers the calcium-induced calcium release of intracellular Ca²⁺ (CICR) from the sarcoplasmic reticulum (SR) through activation of the ryanodine receptor (Ryr), eventually leading to cardiomyocyte contraction (Bers, 2002).

The importance and ubiquity of Ca²⁺ as an intracellular signalling molecule suggests that altered channel function could give rise to widespread cellular and organ defects. Indeed, a variety of cardiovascular diseases, including atrial fibrillation, heart failure, ischemic heart disease, Timothy syndrome, and diabetic cardiomyopathy have been related to alterations in the density or function of the LTCC (Bodi et al., 2005; Mukherjee and Spinale, 1998; Pereira et al., 2006; Quignard et al., 2001). However, the molecular basis for dysregulation of LTCC function and the possible involvement of Akt in I_{Ca,L} (current density of L-type Ca2+ currents) regulation remains unresolved.

Viard et al., 2004, showed that Akt-dependent phosphorylation of the Caᵥβ2 subunit is important in promoting the chaperoning of the Caᵥ2.2 pore-forming unit to the plasma membrane. However, this study focused on neuronal cells and on a particular isoform of Caᵥβ2.

There is still a need in the art, therefore, for ways in which to modulate cardiac contractility. This is useful in treating conditions of where cardiac contractility is impaired such as dilated cardiomyopahty and, in general, cardiac hypertrophy and failure, both primitive and after myocardial infarction.

US 20087/0118438 A1 (Antzelevitch and Pollevick) discloses certain mutations that lead to a loss of function in Calcium Channel peptides, said mutations incurring “sudden cardiac death.” WO 2008/060618 A1 (University of Florida Research Foundation) discloses a method of identifying a subject as having a propensity to have an adverse cardiovascular event by assessing mutations in a number of genes and proteins (alpha-adducin (ADD1) gene, calcium activated potassium channel (KCNMB1) gene, Betal-adrenergic receptor (ADRB1) gene, Beth7-adrenergic receptor (ADRB2) gene, leukotriene A4 hydrolase (LT A4H), arachidonate 5-lipoxygenase-activating protein (ALOX5AP), CACNAIC, CACNB2, and ALOX5 gene) relative to a wild-type reference sequence.

Surprisingly, we have identified a novel post-translational mechanism by which Akt modulates LTCC function under physiological conditions, highlighting the pivotal role of this kinase in cardiac function. In particular, we have found that the pore-forming channel subunit Caᵥα1 contains highly conserved PEST sequences that direct rapid protein degradation. Akt mediated phosphorylation of the Caᵥβ2 LTCC-chaperone subunit prevents PEST site recognition, thereby slowing or preventing Caᵥα1 degradation, thus regulating Ca²⁺ channel function and thus alteration of cardiomyocyte contractile function. Without being bound by theory, we believe that Akt-mediated phosphorylation of the Caᵥβ2 subunit (the LTCC chaperone) at its C-terminal region, in particular the “coiled coil” region of Caᵥβ2, induces a conformational shift in Caᵥβ2. This shift in turn stearically hinders protease access to the PEST sequences of Caᵥα1, which may occur via direct association of either the C-terminal portion of or the whole Caᵥβ2 with cytosolic loops on Caᵥα1 or indirectly through the intervention of known/unknown protein partners.

### SUMMARY OF THE INVENTION

Thus, in a first aspect, the invention provides a Caᵥβ2 peptide or variant thereof, or polynucleotides encoding said peptide or variant, for use the modulation of cardiac inotropism.

The Caᵥβ2 peptide may comprise the full length Caᵥβ2 peptide, provided in SEQ ID NO: 1 (murine) but more preferably that provided in SEQ ID NO: 2, which is human. The Akt consensus site is underlined at positions 500-504 (murine) or 502-507 (human). The serine residue that is phosphorylated by Akt and mutated in preferred embodiments of the invention is highlighted in bold at the C′ terminus of the consensus sequences.

SEQ ID NO: 1 (murine):
MVQSDTSKSPPVAAVAQESQMELLESAAPAGALGAQSYGKGARRKNRFKG
SDGSTSSDTTSNSFVRQGSADSYTSRPSDSDVSLEEDREAVRREAERQAQ
AQLEKAKTKPVAFAVRTNVRYSAAQEDDVPVPGMAISFEAKDFLHVKEKF
NNDWWIGRLVKEGCEIGFIPSPVKLENMRLQHEQRAKQGKFYSSKSGGNS
SSSLGDIVPSSRKSTPPSSAIDIDATGLDAEENDIPANHRSPKPSANSVT
SPHSKEKRMPFFKKTEHTPPYDVVPSMRPVVLVGPSLKGYEVTDMMQKAL
FDFLKHRFEGRISITRVTADISLAKRSVLNNPSKHAIIERSNTRSSLAEV
QSEIERIFELARTLQLVVLDADTINHPAQLSKTSLAPIIVYVKISSPKVL
QRLIKSRGKSQAKHLNVQMVAADKLAQCPPQESFDVILDENQLEDACEHL
ADYLEAYWKATHPPSGNLPNPLLSRTLASSTLPLSPTLASNSQGSQGDQR
PDRSAPRSASQAEEEPCLEPVKKSQHRSSSATHQNHRSGTGRGLSRQETF
DSETQESRDSAYVEPKEDYSHEHVDRYVPHREHNHREETHSSNGHRHRES
RHRSRDMGRDQDHNECIKQRSRHKSKDRYCDKEGEVISKRRNEAGEWNRD
VYIRQ
SEQ ID NO: 2 Human:
MVQRDMSKSPPTAAAAVAQEIQMELLENVAPAGALGAAAQSYGKGARRKN
RFKGSDGSTSSDTTSNSFVRQGSADSYTSRPSDSDVSLEEDREAVRREAE
RQAQAQLEKAKTKPVAFAVRTNVSYSAAHEDDVPVPGMAISFEAKDFLHV
KEKFNNDWWIGRLVKEGCEIGFIPSPVKLENMRLQHEQRAKQGKFYSSKS
GGNSSSSLGDIVPSSRKSTPPSSAIDIDATGLDAEENDIPANHRSPKPSA
NSVTSPHSKEKRMPFFKKTEHTPPYDVVPSMRPVVLVGPSLKGYEVTDMM
QKALFDFLKHRFEGRISITRVTADISLAKRSVLNNPSKHAIIERSNTRSS
LAEVQSEIERIFELARTLQLVVLDADTINHPAQLSKTSLAPIIVYVKISS
PKVLQRLIKSRGKSQAKHLNVQMVAADKLAQCPPELFDVILDENQLEDAC
EHLADYLEAYWKATHPPSSSLPNPLLSRTLATSSLPLSPTLASNSQGSQG
DQRTDRSAPIRSASQAEEEPSVEPVKKSQHRSSSSAPHHNHRSGTSRGLS
RQETFDSETQESRDSAYVEPKEDYSHDHVDHYASHRDHNHRDETHGSSDH
RHRESRHRSRDVDREQDHNECNKQRSRHKSKDRYCEKDGEVISKKRNEAG
EWNRDVYIRQ

Polynucleotides encoding these protein sequences are also envisaged.

However, the Caᵥβ2 peptide preferably comprises the C-terminal portion of Caᵥβ2, for instance that provided as SEQ ID NO: 3 and 4, representing the “coiled-coil” region of the protein, which is particularly preferred.

SEQ ID NO: 3 (murine):
ASSTLPLSPTLASNSQGSQGDQRPDRSAPRSASQAEEEPCLEPVKKSQHR
SSSATHQNHRSGTGRGLSRQETFDSETQESRDSAYVEPKEDYSHEHVDRY
VPHREHNHREETHSSNGHRHRESRHRSRDMGRDQDHNECIKQRSRHKSKD
RYCDKEGEVISKRRNEAGEWNRDVYIRQ
SEQ ID NO: 4 (human):
ATSSLPLSPT LASNSQGSQG DQRTDRSAPI RSASQAEEEP
SVEPVKKSQH RSSSSAPHHN HRSGTSRGLS RQETFDSETQ
ESRDSAYVEP KEDYSHDHVD HYASHRDHNH RDETHGSSDH
RHRESRHRSR DVDREQDHNE CNKQRSRHKS KDRYCEKDGE
VISKKRNEAG EWNRDVYIRQ

It is particularly preferred however that the Caᵥβ2 peptide comprises merely the Akt-consensus sequence, provided as SEQ ID NO: 23 (N′-RTDRS-C′). Preferably, the Caᵥβ2 peptide comprises the coiled coil region of the Caᵥβ2, SEQ ID NO: 3 or 4.

The variant is preferably any mimetic of the Caᵥβ2 peptide that is a functional variant, i.e. capable of modulation of cardiac inotropism. This variant may mimic the phosphorylation of Caᵥβ2 or, alternatively, mimic Caᵥβ2 in its native, un-phosphorylated state. In this regard, the phosphorylated Caᵥβ2 mimic may be considered an agonist of phosphorylated Caᵥβ2, whilst the un-phosphorylated Caᵥβ2 mimic may be considered an antagonist of phosphorylated Caᵥβ2. It will be appreciated that the variant may not only be a peptide but also a synthetic molecule mimicking the effect of a peptide. It is also preferred that these variants may be designed and/or locked into a particular structural conformation to increase the specificity of binding to Caᵥα1, Caᵥβ2 or any other interacting partners.

It is particularly preferred that the peptide or variant is capable of preventing proteolytic degradation of Caᵥα1 and, in particular its PEST sequences. Suitable PEST sequences are provided in Table 1.

It will be appreciated that the terms modulation of cardiac inotropism and cardiac contractility can be interchanged. De-stabilisation of the calcium channel will lead to a reduced calcium flux therethrough and a resulting decrease in cardiac contractility. When Caᵥβ2 is phosphorylated, or a modulator (such as a synthetic molecule) mimicking Caᵥβ2 phosphorylation is provided, then the LTCC is stablised, thereby providing at least basal, and preferably enhanced, cardiac inotropism. Similarly, when the PEST sequences of Caᵥα1 are exposed to the cellular degradation machinery, for instance when Caᵥβ2 is not phosphorylated or a modulator mimicking Caᵥβ2 in its un-phosphorylation state is provided, then the LTCC is de-stablised, thereby providing at least reduced cardiac inotropism.

Suitable agonists will therefore increase cardiac inotropism, whilst a suitable antagonist will decrease cardiac inotropism.

The variant has preferably at least 70% sequence homology, more preferably at least 75% sequence homology, more preferably at least 80% sequence homology, more preferably at least 85% sequence homology, more preferably at least 90% sequence homology, more preferably at least 95% sequence homology, more preferably at least 99% sequence homology and most preferably at least 99.5% sequence homology with SEQ ID NOs 1 and 2. In each case, it is preferred that that the variant comprises the Akt consensus sequence (SEQ ID NO: 3). The same applies for any nucleotide sequences, although it will be appreciated that these may also be capable of hybridizing to the reference sequence under highly stringent conditions, such as washing in 6×SSC. Conservative substitutions are also envisaged in the peptide or nucleotide variants.

Particularly preferred variants are those where the putative phosphorylation site, preferably a Ser or Thr residue has been mutated. Suitable examples include S625A, S625E and: S625D (referring to the numbering in SEQ ID NO: 1) or positions corresponding thereto. Also preferred are variants where the Akt binding site has been mutated or is stearically hindered by the mutation to prevent Akt binding to, and therefore phosphorylating, Caᵥβ2.

The peptide or variants may be conjugated or coupled to another protein, antibody, or any other molecule capable of directing the peptide or variant to a specific cell type (tissue specificity), or non-peptide synthetic molecules mimicking the effects of these peptides. This also encompasses a fusion protein, which can be encoded with the present peptide or variant by the same polynucleotide.

The polynucleotides of the invention may be DNA or RNA or mixtures of both. Preferably, the polynucleotides encode the Caᵥβ2 peptide or variant under the control of a suitable promoter or a system such as the tet operon system that allows the user a degree of control over the expression of the system. Suitable promoters include a cardiac specific promoter (such as myosin heavy chain, Troponin I, for instance), which might be used to redirect the expression specifically to the myocardium.

The polynucleotides may comprise or encode antisense polynucleotides or RNAi, such as siRNA or microRNA. The microRNA may be specific for the 3′UTR of Akt or PDK1, but is most preferably specific for the 3′UTR of Caᵥβ2.

Delivery of the polynucleotides may be via a plasmid or suitable vector, such as an adeno-, retro- or lenti-viral vector. Thus, the invention also provides a vector comprising the polynucleotides encoding the Caᵥβ2 peptide or variant. The vector may be delivered by a “gene-gun,” by electroporation or in the form of a pharmaceutically acceptable formulation. It may be administered to a mucosal lining, for instance orally, nasally or rectally or parentarally (i.e. not through the alimentary canal but rather by injection subcutaneously, intramuscularly, intraorbitally, intracapsularly, intraspinally, intrasternally, or intravenously).

Preferred levels of the peptide for administration and/or expression are in the region of 1-10 mg/kg to 1-10 μg/kg, although this will be readily determined by a physician.

In a further aspect, the invention also provides a pharmaceutical composition comprising or encoding a Caᵥβ2 peptide or polynucleotide, or functional variants thereof. Preferably, the Caᵥβ2 peptide, polynucleotide or variant comprises a mutation in the Akt consensus site, as discussed above, and most preferably corresponding to Ser625 in Caᵥβ2.

It will be appreciated that the Caᵥβ2 peptide or polynucleotide variant has cardiac inotropism/contractility modulating activity.

A further aspect of the invention is a cell, preferably a cardiomyocyte, comprising the present Caᵥβ2 peptide, polynucleotide or variants. The cell has preferably been transformed, by a means of delivery discussed above, to express the Caᵥβ2 peptide, polynucleotide or variants.

In a further aspect, the invention provides a PEST binding factor, such as a protein or polynucleotides, capable of binding to the PEST sequences of Caᵥα1 to prevent degradation thereof. Such a protein may have a large PEG molecule attached thereto or is a glycoprotein, the PEG or sugar unit(s) hindering the access of the PEST degraders.

### BRIEF DESCRIPTION OF THE FIGURES

FIG. 1. Alteration of Ca²⁺ handling proteins in PDK1 KO cardiomyocytes. (A) Western blot and (B) densitometric analyses of ventricular homogenates along a time course of tamoxifen inductions (day 1 to 6 treatment is indicated by the bar) using various antibodies. A representative experiment is shown (n=3). (C) Total Akt activity in WT and KO cardiomyocyte lysates assayed using a GSK313/a Akt-specific substrate.

FIG. 2. Impaired intracellular Ca²⁺ handling and contractility in PDK1 KO cardiomyocytes. (A-B) Smaller Ca²⁺ current in KO cardiomyocytes: (A) Whole-cell representative I_{Ca,L} currents normalized for difference in cell size. (B) I_{Ca,L} I-V current/voltage relationships (n=12) (*P<0.05, **P<0.01). (C-D) Cardiomyocyte contraction and Ca²⁺ transients at different stimulation frequencies. (C) Cardiomyocyte shortening is decreased in KO compared to WT cardiomyocytes (*P<0.05 ANOVA). (D) Ca²⁺-frequency relationship indicates smaller peak systolic but not diastolic Ca²⁺ in KO compared to WT cells (*P<0.05, ANOVA).

FIG. 3. Akt mediates regulation of Caᵥα1 protein density at the plasma membrane. (A) RT-PCR analysis of Caᵥα1 mRNA expression from WT and KO ventricular extracts. GADPH serves as loading control. (B) Western blot analysis of whole lysate, membrane, and microsomal fractions from WT and KO ventricular extracts. (C) YFP-Caᵥα1 transfected COS-7 cells alone or in combination with Caᵥβ2-expression vector were serum-starved and treated with Akt inhibitor and 1 μM bafilomycin-A1, 25 μM MG132, or 25 μM calpeptin. 6 h post drug administration, cell lysates were prepared and subjected to Western blot analysis for YFP. GAPDH served as a loading control. (D-E) Caᵥα1 protein levels in KO cardiomyocytes infected with empty (mock) or active E40K-Akt (AdAkt) expressing adenoviral vector (D) and in whole lysates of WT and E40K-Akt (Tg Akt) hearts (E). Representative experiments are shown (n=4).

FIG. 4. Akt interacts with and phosphorylates Caᵥβ2. (A) Coimmunoprecipitation assay of Akt and Caᵥβ2. Ventricular homogenates from WT and HA-E40K-Akt transgenic mice (Tg Akt) immunoprecipitated with antibodies against HA and immunoblotted for Caᵥβ2 as well as HA as a control. (B) Examination of Caᵥβ2 phosphorylation by Akt. In vitro kinase assays were performed with immunoprecipitated Caᵥβ2 incubated with recombinant active Akt and ³²P labeled ATP (left) or immunoprecipitated Caᵥβ2 from WT and KO cardiac extracts from mice treated or not with insulin (1 mU/g) using PAS (Phospho-Akt Substrate) antibody (right). (C) Back-phosphorylation assay of Caᵥβ2 from WT and KO hearts. Immunoprecipitated Caᵥβ2 from solubilized membranes was in vitro back-phosphorylated using recombinant active Akt and [γ³²]ATP. Precipitate amounts were assayed for ³²PCaᵥβ2 and total Caᵥβ2. Representative experiments are shown (n=4).

FIG. 5. Akt phosphorylation of Caᵥβ2 protects Caᵥα1 from protein degradation. (A-C) YFP-Caᵥα1 co-transfected 293T cells with the indicated mutant variant of Caᵥβ2. Cells were serum-starved overnight and treated with (A) 100 μM insulin or (B-C) 5 μM Akt inhibitor as indicated. The expression of YFP-Caᵥα1 in lysates was monitored by Western blot analysis with anti-YFP antibody, and normalized based on transfection efficiency (Caᵥ(32) and protein amount (Tubulin) (n=3). (D) Caᵥα1 and Caᵥβ2 co-transfected 293T cells were treated with siAkt expressing vector as indicated. 3 days post transfection, cell lysate was tested by Western blot analysis. Protein loading was normalized to GAPDH levels. Representative experiments are shown (n=3).

FIG. 6. Akt phosphorylation of Caᵥβ2 preserves Caᵥα1 currents. Ca²⁺ currents recorded in cotransfected tsA-201 cells with YFP-Caᵥα1 and either Caᵥβ2-WT, Caᵥβ2-SE, or Caᵥβ2-SA mutant, cultivated for 36 h in the presence or absence of fetal bovine serum (10%). Currents were recorded 1-2 minutes after the whole-cell configuration was achieved (i.e. after stabilization of the current) and were elicited by a 0 mV depolarization of 200 ms duration applied from a holding potential of −80 mV. Currents are normalized to cell capacitance (Current density, pA/pF). Representative current traces are shown. n>35 at each condition, (*P<0.05 compared to YFP-Caᵥα1, ANOVA).

FIG. 7. Rapid-protein-degradation PEST sequences determine Caᵥα1 protein instability. (A) Schematic representation of Caᵥα1 mapping the α1-interacting domain (AID) and PEST sequences in the I-II and II-III cytosolic loops. Deleted PEST sequences (P, H) are highlighted in red. (B) Western blot and immunofluorescence analyses showing relative levels of WT and PEST deleted mutants of YFP-Caᵥα1 (n=3). Bar represents 5 μm. (C) Wild-type Caᵥα1 subunit (alone or cotransfected with Caᵥβ2SE) and its in-frame ΔPEST mutants (Caᵥα1-ΔP and Caᵥα1-ΔH) half-lives were determined in COST cells. After overnight starvation, transfected cells were pulse-chased and analyzed along a time course (*P<0.001 compared to Caᵥα1, ANOVA; n=3). (D) Western blot and immunofluorescence analyses showing relative levels of WT GFP and N-terminal fusion PEST mutants (n=3). Bar represents 20 μm. (E) The Caᵥα1 C-terminus interacts with the Akt-phosphorylated-GST-Caᵥβ2 coiled coil region. Bacterially expressed GST or GST-C-Caᵥβ2 (Caᵥβ2: aa 480-655) fusion protein and glutathione sepharose beads were incubated with equal amounts of in vitro translated [³⁵S] methionine-labeled C-Caᵥα1 (Caᵥα1: aa 1477-2169). Binding occurred only with Akt-phosphorylated GST-C-Caᵥβ2. Bound proteins were resolved by SDS-PAGE (4-12%). 10% of the input protein in each binding reaction is shown. Coomassie staining of SDS-PAGE is shown in the bottom panel. (F) Ca²⁺ currents recorded in tsA-201 cells cotransfected with Caᵥβ2-WT and either Caᵥα1-WT or Caᵥα1-ΔH and cultivated for 36 h in the presence or absence of fetal bovine serum (10%). Current densities (pA/pF) are normalized to the control condition. n>35 at each condition, (*P<0.05 compared to Caᵥα1, ANOVA).

FIG. 8. Proposed mechanism. Akt, followed by PDK1 activation, phopshorylates Caᵥβ2 at the C-terminal coiled-coil domain. The phosphorylation allows association of the C-terminal portion of Caᵥβ2 with the Caᵥα1 C-terminal domain. A conformation shift, in turns, prevents PEST sequence recognition, stabilizing Caᵥα1 protein levels. Blue and red ribbon in Caᵥα1 represent AID and PEST sequences, respectively.

FIG. 9. Characterization of mice lacking PDK1 expression. (A) PDK protein and RNA levels assessed by Western blot (upper) and RT-PCR (lower) analyses of atria, left (LV), and right (RV) ventricular cardiomyocytes from WT and KO mice. Protein and RNA loading was normalized to GAPDH levels, respectively. (B) Immunofluorescence staining of cardiomyocytes isolated from WT and KO hearts labeled with antibody against PDK1 (green) and counterstained with Hoechst nuclear stain (blue). Bar represent 15 μm. (C) Survival curve for mice lacking PDK1 (KO) in the heart. Mortality begins 5 days after tamoxifen injection and reaches 100% by day 10 after beginning of treatment. Time points of tamoxifen injections and echocardiography analysis (arrows) are shown (n=10). (D) Echocardiographic (M-mode) assessment of left ventricular size and function. Left ventricular diastolic internal dimensions LVIDd (red bar) and left ventricular systolic internal dimensions LVIDs (blue bar) were increased in KO mice. Heart rates were 486 and 511 bpm, respectively. (E) H&E-stained paraffin sections show severe dilatation and thinning of KO hearts. (F) Western blot analysis for caspase 3 activation in WT and KO heart homogenates. Basal apoptotic activation as a consequence of tamoxifen treatment was also observed in WT (as previously observed in supplementary ref (Zartman et al., 2004)). Amounts of loaded protein were verified with tubulin antibodies. (G) Representative Masson's trichrome staining of tissue sections from WT and KO hearts.

FIG. 10. Regulation of Ca²⁺ handling proteins by Akt. (A) Western blot analysis of ventricular homogenates from WT and KO mice. (B) Western blot analysis of ventricular homogenates along a time course of tamoxifen inductions (day 1 to 6 treatment is indicated by the bar) using various antibodies. A representative experiment is shown (n=3).

FIG. 11. Altered intracellular calcium handling in PDK1 KO cardiomyocytes. (A) Representative Ca²⁺ traces are shown for WT (upper) and KO (lower) cardiomyocytes. (B) Reduced averaged twitch Ca²⁺ transient amplitude is shown in KO compared to WT cardiomyocytes (left, unpaired t test, P<0.05). No difference was found in total SR Ca²⁺ analysis (right). WT and KO results are shown in blue and red respectively.

FIG. 12. Caᵥβ2 interacts with Akt isoforms and Akt affects Caᵥα1 protein stability. (A) Immunoprecipitated Akt isoforms from WT cardiac extracts from mice treated or not with insulin (1 mU/g) assayed for Caᵥβ2. Input protein in each co-immunoprecipitation is shown. (B) Caᵥα1, Caᵥα1-ΔP, or Caᵥα1-ΔH co-transfected 293T cells with either Caᵥβ2 or Caᵥβ2-SE were infected with indicated active (AdAkt) or dominant negative (AdAktDN) Akt expressing adenoviral vectors. Cells were serum-starved overnight as indicated. The expression of Caᵥα1 and phosphorylation of GSK in lysates was monitored by Western blot analysis. (C) Caᵥα1, Caᵥα1-ΔP, or Caᵥα1 co-transfected 293T cells with either Caᵥβ2 or Caᵥβ2-SE were treated with siAkt expressing vector as indicated. 3 days post transfection, cells were lysated for protein extraction. Protein loading was normalized to GAPDH levels. Representative experiments are shown (n=3).

FIG. 13. Serum deprivation and PEST-H deletion does not modify steady-state activation parameters. Ca²⁺ currents recorded in cotransfected tsA-201 cells with Caᵥβ2-WT and either Caᵥα1-WT or Caᵥα1-OH, and cultivated for 36 h in the presence or absence of fetal bovine serum (10%). IV curves are normalized to the maximal current. n>35 at each condition, (ANOVA).

### DETAILED DESCRIPTION OF THE INVENTION

The present inventors have also shown through interaction experiments that phosphorylated—Caᵥβ2 binds only to Caᵥα1 C-terminal tail. Without being bound by theory, therefore, they hypothesize that the binding induces conformational changes in the alpha1 protein, thus covering PEST sequences. Therefore, the present invention encompasses any peptide or variant that binds the Caᵥα1 C-terminal tail region.

By degradation, it will be appreciated that this refers to proteolytic action, i.e. cleavage of the PEST sequences by cellular machinery, such as proteosomes.

It is particularly preferred that the variant is capable of preventing proteolytic degradation of Caᵥα1 and, in particular its PEST sequences. Suitable PEST sequences are any of provided in Table 1.

Also provided are the Caᵥβ2 peptide or variant or polynucleotides for use in therapy, and the Caᵥβ2 peptide or variant or polynucleotides for use in treating a condition associated with, or treatable by, cardiac contractility modulation. Methods of treatment or prophylaxis comprising administering the Caᵥβ2 peptide or variant or polynucleotides to a patient in need thereof are also provided. Preferred conditions are those associated with cardiac inotropism or cardiac contractility. Preferred examples include dilated cardiomyopahty and cardiac hypertrophy and failure, both primitive and after myocardial infarction. As mentioned above, it will be appreciated that this extends to synthetic molecules.

The invention also provides Caᵥα1 variants in which either the I-II (Caᵥα1-ΔP) or II-III (Caᵥα1-ΔH) cytosolic linker region of Caᵥα1 has been mutated, preferably by an in-frame deletion. These Caᵥα1 mutants at lack one or more, and preferably at least 50% and more preferably at least 75% or even all their PEST sequences. They may be delivered in the same manner as discussed above.

P sequence (mouse):

| | |
|---|---|
| KGYLDWITQAEDIDPENEDEGMDEDK | (SEQ ID NO: 18) |
| P sequence (human): | |
| KGYLDWITQAEDIDPENEDEGMDEEK | (SEQ ID NO: 19) |
| H sequence (mouse and human): | |
| GEEDEEEPEMPVGPR | (SEQ ID NO: 20) |

SEQ ID NO: 21 is the Caᵥα1-ΔH (mouse) sequence, wherein the H sequence (to be removed) is underlined and placed in bold (at positions 844-858 below):

MVNENTRMYVPEENHQGSNYGSPRPAHANMNANAAAGLAPEHIPTPGAAL
SWQAAIDAARQAKLMGSAGNATISTVSSTQRKRQQYGKPKKQGGTTATRP
PRALLCLTLKNPIRRACISIVEWKPFEIIILLTIFANCVALAIYIPFPED
DSNATNSNLERVEYLFLIIFTVEAFLKVIAYGLLFHPNAYLRNGWNLLDF
IIVVVGLFSAILEQATKADGANALGGKGAGFDVKALRAFRVLRPLRLVSG
VPSLQVVLNSIIKAMVPLLHIALLVLFVIIIYAIIGLELFMGKMHKTCYN
QEGIIDVPAEEDPSPCALETGHGRQCQNGTVCKPGWDGPKHGITNFDNFA
FAMLTVFQCITMEGWTDVLYWMQDAMGYELPWVYFVSLVIFGSFFVLNLV
LGVLSGEFSKEREKAKARGDFQKLREKQQLEEDLKGYLDWITQAEDIDPE
NEDEGMDEDKPRNMSMPTSETESVNTENVAGGDIEGENCGARLAHRISKS
KFSRYWRRWNRFCRRKCRAAVKSNVFYWLVIFLVFLNTLTIASEHYNQPH
WLTEVQDTANKALLALFTAEMLLKMYSLGLQAYFVSLFNRFDCFIVCGGI
LETILVETKIMSPLGISVLRCVRLLRIFKITRYWNSLSNLVASLLNSVRS
IASLLLLLFLFIIIFSLLGMQLFGGKFNFDEMQTRRSTFDNFPQSLLTVF
QILTGEDWNSVMYDGIMAYGGPSFPGMLVCIYFIILFICGNYILLNVFLA
IAVDNLADAESLTSAQKEEEEEKERKKLARTASPEKKQEVMEKPAVEESK
EEKIELKSITADGESPPTTKINMDDLQPSENEDKSPHSNPDTAGEEDEEE
PEMPVGPRPRPLSELHLKEKAVPMPEASAFFIFSPNNRFRLQCHRIVNDT
IFTNLILFFILLSSISLAAEDPVQHTSFRNHILGNADYVFTSIFTLEIIL
KMTAYGAFLHKGSFCRNYFNILDLLVVSVSLISFGIQSSAINVVKILRVL
RVLRPLRAINRAKGLKHVVQCVFVAIRTIGNIVIVTTLLQFMFACIGVQL
FKGKLYTCSDSSKQTEAECKGNYITYKDGEVDHPIIQPRSWENSKFDFDN
VLAAMMALFTVSTFEGWPELLYRSIDSHTEDKGPIYNYRVEISIFFIIYI
IIIAFFMMNIFVGFVIVTFQEQGEQEYKNCELDKNQRQCVEYALKARPLR
RYIPKNQHQYKVWYVVNSTYFEYLMFVLILLNTICLAMQHYGQSCLFKIA
MNILNMLFTGLFTVEMILKLIAFKPKHYFCDAWNTFDALIVVGSIVDIAI
TEVHPAEHTQCSPSMSAEENSRISITFFRLFRVMRLVKLLSRGEGIRTLL
WTFIKSFQALPYVALLIVMLFFIYAVIGMQVFGKIALNDTTEINRNNNFQ
TFPQAVLLLFRCATGEAWQDIMLACMPGKKCAPESEPSNSTEGETPCGSS
FAVFYFISFYMLCAFLIINLFVAVIMDNFDYLTRDWSILGPHHLDEFKRI
WAEYDPEAKGRIKHLDVVTLLRRIQPPLGFGKLCPHRVACKRLVSMNMPL
NSDGTVMFNATLFALVRTALRIKTEGNLEQANEELRAIIKKIWKRTSMKL
LDQVVPPAGDDEVTVGKFYATFLIQEYFRKFKKRKEQGLVGKPSQRNALS
LQAGLRTLHDIGPEIRRAISGDLTAEEELDKAMKEAVSAASEDDIFRRAG
GLFGNHVTYYQSDSRGNFPQTFATQRPLHINKTGNNQADTESPSHEKLVD
STFTPSSYSSTGSNANINNANNTALGRFPHPAGYSSTVSTVEGHGPPLSP
AVRVQEAAWKLSSKRCHSRESQGATVNQEIFPDETRSVRMSEEAEYCSEP
SLLSTDMFSYQEDEHRQLTCPEEDKREIQPSPKRSFLRSASLGRRASFHL
ECLKRQKDQGGDISQKTALPLHLVHHQALAVAGLSPLLQRSHSPTTFPRP
CPTPPVTPGSRGRPLRPIPTLRLEGAESSEKLNSSFPSIHCSSWSEETTA
CSGSSSMARRARPVSLTVPSQAGAPGRQFHGSASSLVEAVLISEGLGQFA
QDPKFIEVTTQELADACDMTIEEMENAADNILSGGAQQSPNGTLLPFVNC
RDPGQDRAVVPEDESCAYALGRGRSEEALADSRSYVSNL

SEQ ID NO: 22 is the Caᵥα1-ΔH (human) sequence, wherein the H sequence (to be removed) is underlined and placed in bold (at positions 841-855 below):

MVNENTRMYIPEENHQGSNYGSPRPAHANMNANAAAGLAPEHIPTPGAAL
SWQAAIDAARQAKLMGSAGNATISTVSSTQRKRQQYGKPKKQGSTTATRP
PRALLCLTLKNPIRRACISIVEWKPFEIIILLTIFANCVALAIYIPFPED
DSNATNSNLERVEYLFLIIFTVEAFLKVIAYGLLFHPNAYLRNGWNLLDF
IIVVVGLFSAILEQATKADGANALGGKGAGEDVKALRAFRVLRPLRLVSG
VPSLQVVLNSIIKAMVPLLHIALLVLFVIIIYAIIGLELFMGKMHKTCYN
QEGIAAEDDPSPCALETGHGRQCQNGTVCKPGWDGPKHGITNFDNFAFAM
LTVFQCITMEGWTDVLYWVNDAVGRDWPWIYFVTLIIIGSFFVLNLVLGV
LSGEFSKEREKAKARGDFQKLREKQQLEEDLKGYLDWITQAEDIDPENED
EGMDEEKPRNMSMPTSETESVNTENVAGGDIEGENCGARLAHRISKSKFS
RYWRRWNRFCRRKCRAAVKSNVFYWLVIFLVFLNTLTIASEHYNQPNWLT
EVQDTANKALLALFTAEMLLKMYSLGLQAYFVSLFNRFDCFVVCGGILET
ILVETKIMSPLGISVLRCVRLLRIFKITRYWNSLSNLVASLLNSVRSIAS
LLLLLFLFIIIFSLLGMQLFGGKFNFDEMQTRRSTFDNFPQSLLTVFQIL
TGEDWNSVMYDGIMAYGGPSFPGMLVCIYFIILFICGNYILLNVFLAIAV
DNLADAESLTSAQKEEEEEKERKKLARTASPEKKQELVEKPAVGESKEEK
IELKSITADGESPPATKINMDDLQPNENEDKSPYPNPETTGEEDEEEPEM
PVGPRPRPLSELHLKEKAVPMPEASAFFIFSSNNRFRLQCHRIVNDTIFT
NLILFFILLSSISLAAEDPVQHTSFRNHILFYFDIVFTTIFTIEIALKMT
AYGAFLHKGSFCRNYFNILDLLVVSVSLISFGIQSSAINVVKILRVLRVL
RPLRAINRAKGLKHVVQCVFVAIRTIGNIVIVTTLLQFMFACIGVQLFKG
KLYTCSDSSKQTEAECKGNYITYKDGEVDHPIIQPRSWENSKFDFDNVLA
AMMALFTVSTFEGWPELLYRSIDSHTEDKGPIYNYRVEISIFFIIYIIII
AFFMMNIFVGFVIVTFQEQGEQEYKNCELDKNQRQCVEYALKARPLRRYI
PKNQHQYKVWYVVNSTYFEYLMFVLILLNTICLAMQHYGQSCLFKIAMNI
LNMLFTGLFTVEMILKLIAFKPKGYFSDPWNVFDFLIVIGSIIDVILSET
NPAEHTQCSPSMNAEENSRISITFFRLFRVMRLVKLLSRGEGIRTLLWTF
IKSFQALPYVVLLIVMLFFIYAVIGMQVFGKIALNDTTEINRNNNFQTFP
QAVLLLFRCATGEAWQDIMLACMPGKKCAPESEPSNSTEGETPCGSSFAV
FYFISFYMLCAFLIINLFVAVIMDNFDYLTRDWSILGPHHLDEFKRIWAE
YDPEAKGRIKHLDVVTLLRRIQPPLGFGKLCPHRVACKRLVSMNMPLNSD
GTVMFNATLFALVRTALRIKTEGNLEQANEELRAIIKKIWKRTSMKLLDQ
VVPPAGDDEVTVGKFYATFLIQEYFRKFKKRKEQGLVGKPSQRNALSLQA
GLRTLHDIGPEIRRAISGDLTAEEELDKAMKEAVSAASEDDIFRRAGGLF
GNHVSYYQSDGRSAFPQTFTTQRPLHINKAGSSQGDTESPSHEKLVDSTF
TPSSYSSTGSNANINNANNTALGRLPRPAGYPSTVSTVEGHGPPLSPAIR
VQEVAWKLSSNRCHSRESQAAMAGQEETSQDETYEVKMNHDTEACSEPSL
LSTEMLSYQDDENRQLTLPEEDKRDIRQSPKRGFLRSASLGRRASFHLEC
LKRQKDRGGDISQKTVLPLHLVHHQALAVAGLSPLLQRSHSPASFPRPFA
TPPATPGSRGWPPQPVPTLRLEGVESSEKLNSSFPSIHCGSWAETTPGGG
GSSAARRVRPVSLMVPSQAGAPGRQFHGSASSLVEAVLISEGLGQFAQDP
KFIEVTTQELADACDMTIEEMESAADNILSGGAPQSPNGALLPFVNCRDA
GQDRAGGEEDAGCVRARGRPSEEELQDSRVYVSSL

Where reference is made herein to a particular position, it will be appreciated that this also refers to the equivalent position of such a feature or motif in a similar or variant sequence.

In short, the insulin IGF-1/PI3K/Akt signalling pathway has been suggested to improve cardiac inotropism and increase Ca²⁺ handling through the effects of the protein kinase Akt. However, to date the even the basic underlying molecular mechanisms behind the function of the myocyte Calcium channel remain largely unknown. However, we have found that Akt has an unanticipated regulatory function in controlling L-type Ca²⁺ channel (LTCC) protein density. Furthermore, we have surprisingly found that the pore-forming channel subunit Caᵥα1 contains highly conserved PEST sequences (signals for rapid protein degradation). In-frame deletion of these PEST sequences result in increased Caᵥα1 protein levels. Our findings show that Akt-dependent phosphorylation of Caᵥβ2, the LTCC chaperone for Caᵥα1, antagonizes Caᵥα1 protein degradation by preventing Caᵥα1-PEST sequence recognition. This leads to increased LTCC density and consequent modulation of Ca²⁺ channel function. This novel mechanism by which Akt modulates LTCC stability could profoundly influence cardiac myocyte Ca²⁺ entry, Ca²⁺ handling, and contractility.

Without being bound by theory, we believe that Akt-mediated phosphorylation of the Caᵥβ2 subunit, i.e. the LTCC chaperone, at its C-terminal region, in particular the “coiled coil” region of Caᵥβ2, induces a conformational shift in Caᵥβ2. This shift in turn stearically hinders protease access to the PEST sequences of Caval, which may occur via direct association of either the C-terminal portion of or the whole Caᵥβ2 with cytosolic loops on Caᵥα1 or indirectly through the intervention of protein partners.

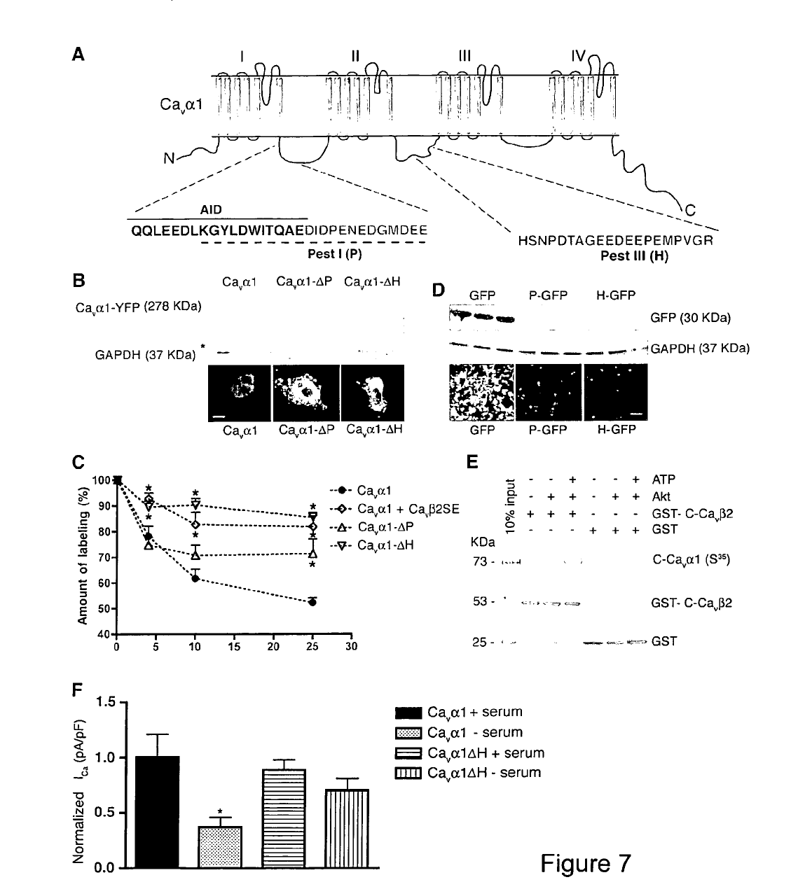

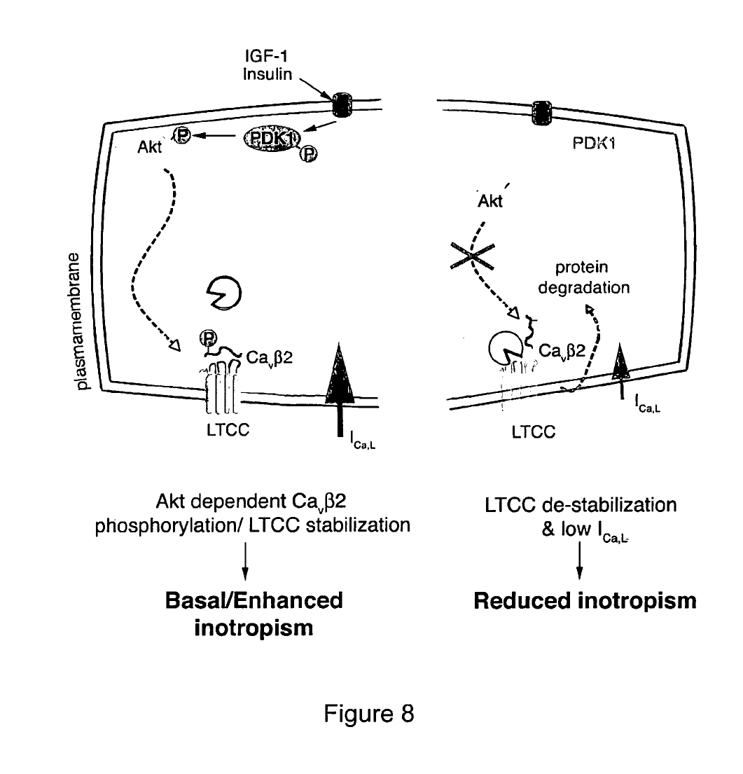

This study reveals a mechanism through which the insulin IGF-1/PI3K/PDK1/Akt pathway can sustain or modulate Ca²⁺ entry in cardiac cells via the voltage-gated LTCC and eventually affect cardiac contractility. Using a mouse model with an inducible and cardiomyocyte-specific deletion of the upstream activator PDK1, we showed that Akt is of key importance for the structural organization and functionality of the LTCC complex at the plasma membrane. This regulation of LTCC activity is directly related to the Akt-mediated phosphorylation of the accessory subunit Caᵥβ2, which in turn results in increased protein density of the pore-forming Caᵥα1 subunit through protection of PEST sequences from the proteolytic degradation system. In the absence of phosphorylated Akt, the Ca²⁺ current is reduced, resulting in depressed Ca²⁺ transient and contractility. It is therefore tempting to speculate that the Akt-mediated phosphorylation of Caᵥβ2 and the consequent direct association of Caᵥβ2 C-terminal tail with the Caᵥα1 C-terminal coiled-coil region (FIG. 7E) may induce conformational changes that prevent PEST sequences to be recognized by the cell degradation system (FIG. 8).

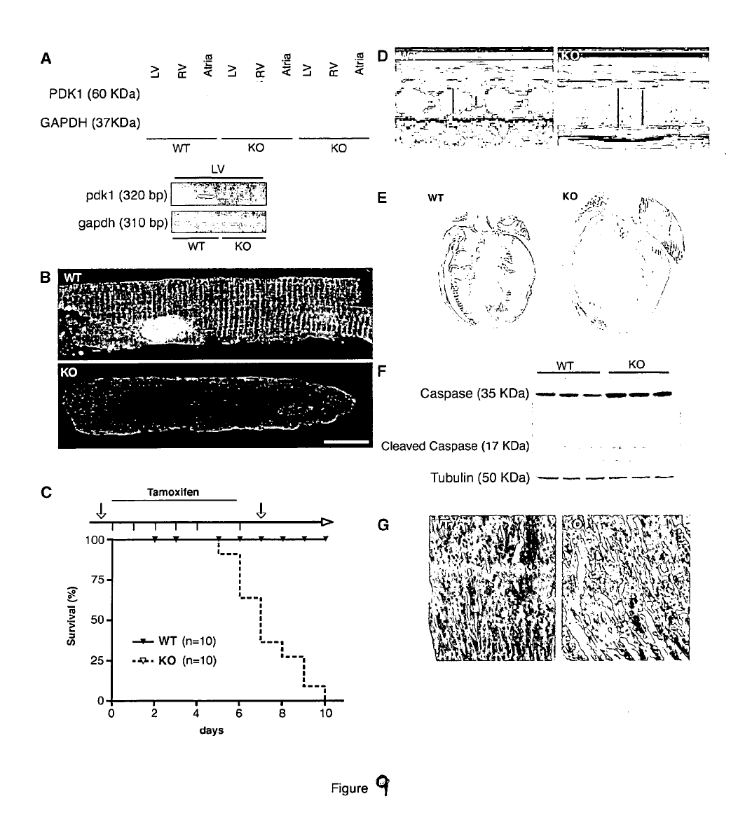

The identified mechanism alone is unlikely to be responsible for the detrimental cardiac defects observed in the PDK1 KO mouse model. To assess whether a reduction in the Akt anti-apoptotic activity could lead to increased cell death, we measured caspase 3 activation (FIG. 9). However, consistent with previous evidence reported by Alessi's group (Mora et al., 2003), our results failed to prove any significant involvement of this mechanism in the PDK1 KO phenotype. Our PDK1 KO mouse model does not appear to progress through slow transitional states typical of heart failure but rather progresses directly to a dilated cardiac phenotype, which eventually leads to premature death (FIG. 9). Therefore, we hypothesize that the lethal phenotype is caused by activation of more complex systems that rapidly remodel the extracellular matrix and cell-to-cell contacts, and change the energy metabolism. Further studies are required to unravel the complex mechanisms that contribute to the establishment of the observed PDK1 KO mice heart phenotype.

Several findings have shown the importance of the insulin IGF-1/PI3K/Akt pathway in heart function. Our group has previously demonstrated that overexpression of an active form of Akt1 results in improved cardiac inotropism both in vivo (Condorelli et al., 2002) and in vitro (Kim et al., 2003), augmenting I_{Ca,L}. Similar results were recently obtained in a mouse model with cardiac specific Akt1 nuclear-overexpression (Rota et al., 2005) and in mice deficient for PTEN (Phosphatase and TENsin homolog deleted on chromosome 10), an antagonizer of PI3K activity (Sun et al., 2006). In addition, short-term administration of IGF-1 in animal studies has also been reported to increase cardiac contractility (Duerr et al., 1995).

However, the mechanism through which the insulin IGF-1/PI3K/Akt pathway affects Ca²⁺ current has remained elusive. In an elegant in vitro study, Viard and coworkers (Viard et al., 2004) demonstrated that a region of the Caᵥβ2a subunit is involved in the PI3K-induced chaperoning of Caᵥ2.2α in neurons. This PI3K-induced regulation was shown to be mediated by Akt phosphorylation of the Caᵥβ2a subunit, which in turn regulates Caᵥ2.2α, trafficking from the ER to the plasma membrane.

Notably, the C-terminal region containing the putative Akt-phosphorylation consensus site is conserved in all variants of the Caᵥβ2 subunit both in neurons and heart (Viard et al., 2004), thus illustrating the importance of this site. Interestingly, two very short human cardiac splice isoforms, Caᵥβ2f and Caᵥβ2g with preserved Akt-site have been shown to be essential for modulating Ca²⁺ channel function and Caᵥα1 channel density (De Waard et al., 1994; Kobrinsky et al., 2005).

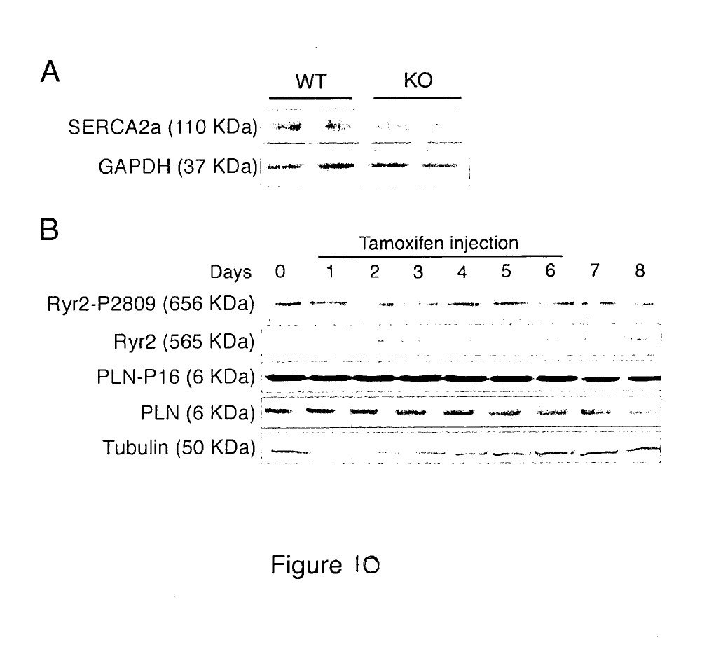

Strikingly, the same two Caᵥβ2 variants do not contain the protein kinase PKA phosphorylation site (Kamp and Hell, 2000), consistent with our data suggesting no PKA involvement in the modulation of LTCC density (FIG. 10B). As a corollary, the presence of this conserved C-terminal region in all Caᵥβ2 splice isoforms corroborates the relevance of identifying new functional motifs that may give important insights into LTCC modulation. Consistent with an important functional role of the conserved Caᵥβ2 C-terminal region, Soldatov and coworkers recently showed that, in the absence of the main Caᵥβ2 protein domain, the selected C-terminal essential determinant (CED) is sufficient for I_{Ca,L} stimulation (Lao et al., 2008). All together, this evidence supports the notion that this region is a potential pharmacological target.

In conclusion, we show that the insulin IGF-1/PI3K/PDK1/Akt pathway regulates Caᵥβ2 chaperone activity through phosphorylation by Akt and suggest that this in turn controls Caᵥα1 channel density by protection of Caᵥα1 from PEST-dependent protein degradation (FIG. 8). This paradigm highlights an unanticipated regulatory function for Akt in modulating LTCC function and provides evidence for an essential role of Akt in the control of cardiomyocyte Ca²⁺ handling and contractility. Interestingly, the high level of conservation of PEST sequences in the Caᵥα1 subunit throughout evolution (Table 1) indicates that our proposed mechanism may play a universal role in regulating cell Ca²⁺ handling and survival. Since pathophysiological states are often accompanied by alterations in LTCC function (Mukherjee and Spinale, 1998), the elucidation of this novel regulatory pathway may open new therapeutic perspectives.

The invention will now be described in more detail with reference to the following examples.

### EXAMPLES

To gain insight into the mechanism of action by which Akt regulates I_{Ca,L} and Ca²⁺ handling in the heart, we studied a mouse line with tamoxifen-inducible (Sohal et al., 2001) and cardiac-specific deletion of PDK1, the upstream activator of all three Akt isoforms. Mice in which exon 3 and 4 of the pdk1 gene were flanked by loxP excision sequences (previously described by Lawlor (Lawlor et al., 2002)) were crossed with transgenic mice expressing an inducible and cardiac-specific MerCreMer α-MHC promoter driving the cre recombinase gene (Sohal et al., 2001), resulting in MerCreMer α-MHC PDK1 mice (KO).

As opposed to the previously described muscle creatine kinase-Cre PDK1 mouse model (Mora et al., 2003) where PDK1 is deleted embryonically in all striated muscles, this model allows for specific deletion of PDK1 in adult heart. A further advantage of this model is the inducible cardiac specific deletion that was necessary to circumvent the embryonic lethality we observed in a mouse model with constitutive α-MHC-Cre cardiac deletion of PDK1 (JHB unpublished data). Similar to the muscle creatine kinase-Cre PDK1 mouse model (Lawlor et al., 2002), PDK1 gene deletion in the adult mouse heart (KO) (FIG. 9A-B) resulted in a lethal phenotype with a mortality that reached 100% at 10 days after tamoxifen injection (FIG. 9C). Age-matched littermate control mice without cre (wildtype (WT)) were unaffected by tamoxifen treatment.

Consistent with findings from the previously reported analysis of the PDK1 KO mouse model (Lawlor et al., 2002), cardiac function evaluated by echocardiography at 7 days after tamoxifen injection, revealed dramatically impaired systolic function with severe dilated cardiomyopathy and an abrupt drop in fractional shortening in KO, but not in WT mice (FIG. 9D, Table 2, and data not shown). Histological examination substantiated the echocardiographic findings, revealing dilatation of both ventricles and atria (FIG. 9E) with apparently no evidence of significant apoptosis or interstitial fibrosis (FIG. 9F-G). These observations indicate that PDK1/Akt activity plays a major role in maintaining adult heart function.

### Deficiency in Akt Activity Leads to a Reduction in the Caᵥα1 Protein Level

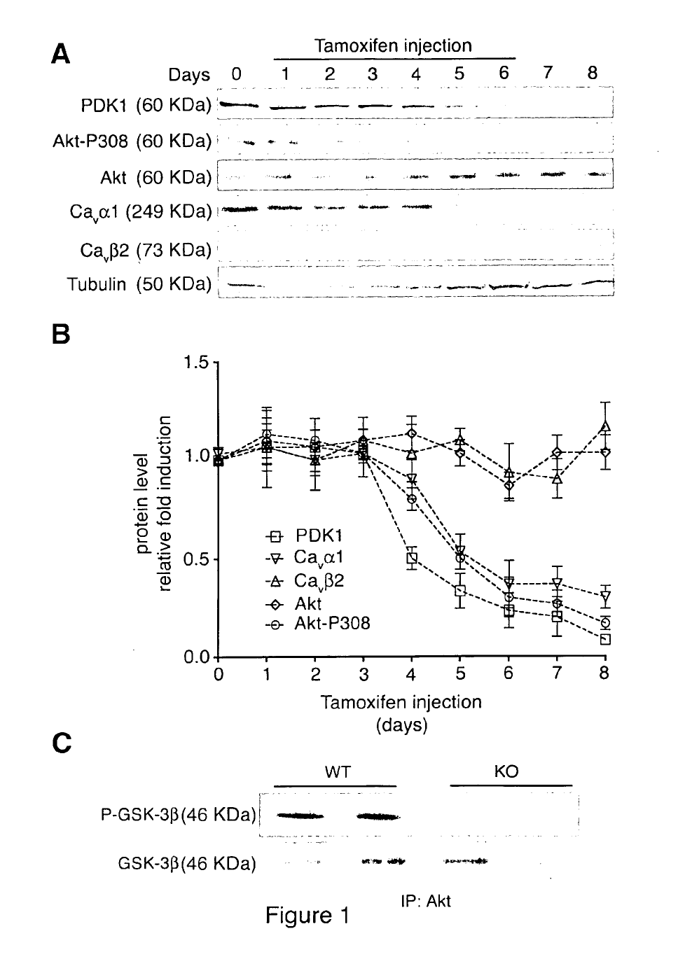

Using the cardiac specific PDK1 knockout mouse model, we investigated whether deficiency in Akt activity affects the expression or activation of signaling molecules that are implicated in Ca²⁺ handling and cardiac function. A time-course analysis of extracts from WT and KO mouse ventricle revealed striking changes in protein expression upon induction of the PDK1 knockout (FIG. 1A-B). Notably, KO mice had decreased protein levels of the pore-forming Ca²⁺ channel subunit (Caᵥα1), which progressed as PDK1 protein expression gradually declined. No change in the protein level of the regulatory Caᵥβ2 subunit was observed. As PDK1 expression decayed, levels of Akt activation also dramatically decreased (assessed by phosphorylation of Akt at the PDK1 phosphorylation site, Thr308), despite unaltered expression of total Akt protein (FIG. 1A-B). Furthermore, Akt activity (assessed using GSK-313 as a substrate) was virtually absent in KO hearts (FIG. 1C). Based on this evidence, we decided to perform further experiments by day 6 after the beginning of treatment.

Although the main physiological action of PDK1 is on Akt activation, PDK1 can potentially influence other members of the cAMP-dependent, cGMP-dependent, and protein kinase C (AGC) kinase protein family, such as PKC and PKA, which could also affect the cellular Ca²⁺ handling (Mora et al., 2004; Williams et al., 2000). PKC activity was, however, unchanged in KO mice (1.15±0.05 fold over WT, not statistically significant, assessed by an assay using a PKC specific peptide as substrate). There was no apparent effect of PDK1 deletion on SERCA2a (FIG. 10A) as well as PKA activity, since the phosphorylation of specific PKA regulatory sites in two SR Ca²⁺-regulatory proteins, ryanodine receptor (Ryr2-P2809) and phospholamban (PLN-P16), were unchanged in KO mice (FIG. 10B), although it cannot be excluded that typical changes associated with heart failure and secondary to adrenergic receptor hyperactivation may take place at subsequent time points. Taken together, these data suggest that an acute reduction in Akt activation affects expression of proteins involved in the Ca²⁺ influx into the cell.

### Deficiency in Akt Activity Affects I_{Ca,L}

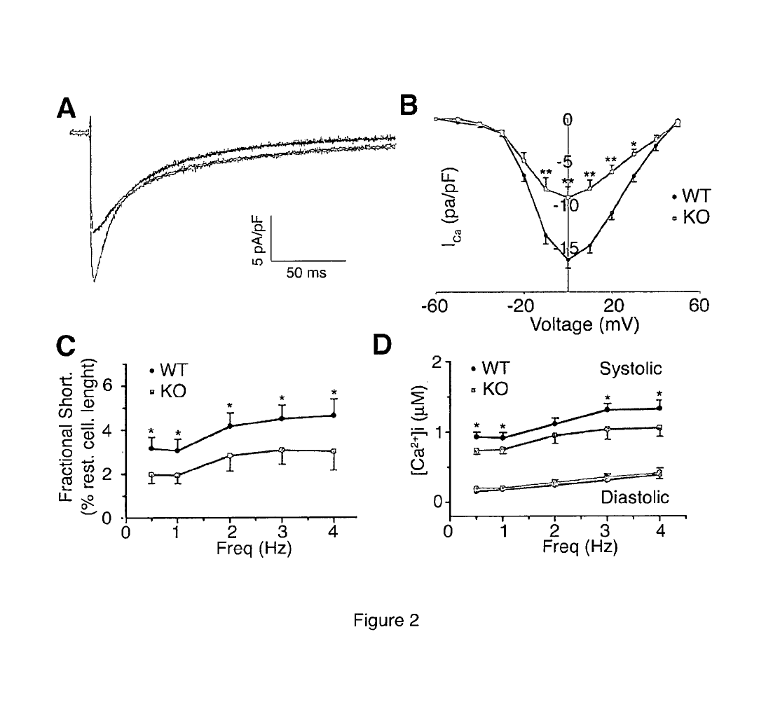

Ca²⁺ handling and inotropism were examined in adult cardiomyocytes freshly isolated from WT and KO mice. Using the whole-cell voltage-clamp technique, we recorded and analyzed LTCC I_{Ca,L} properties. No difference in cell size was observed between WT and KO cells as deduced from membrane capacitance (Mc) measurements. Mc was 116±6 pF in WT cells (n=18) and 115±6 pF in KO cells (n=18). However, the density of I_{Ca,L} (pA/pF) was decreased in KO vs. WT (FIG. 2B). At 0 mV, the density of I_{Ca,L} was −9.08±0.96 pA/pF in KO cells (n=12) vs. −16.26±0.96 pA/pF in WT cells (n=12; p<0.001).

In addition, there was no significant difference in either steady-state activation or inactivation curves (data not shown). Indeed, mean half activation occurred at −12.97±0.53 mV in WT cells vs. −15.07±0.66 mV in KO cells and mean half inactivation occurred at −31.11±0.48 mV in WT cells vs. −30.77±0.42 mV in KO cells. The absence of a shift in the voltage-dependence of these properties (FIG. 2B) was consistent with the absence of modification in gating properties of the LTCC, suggesting that a reduction in the number of functional LTCCs can account for the observed decrease in I_{Ca,L} in KO mice.

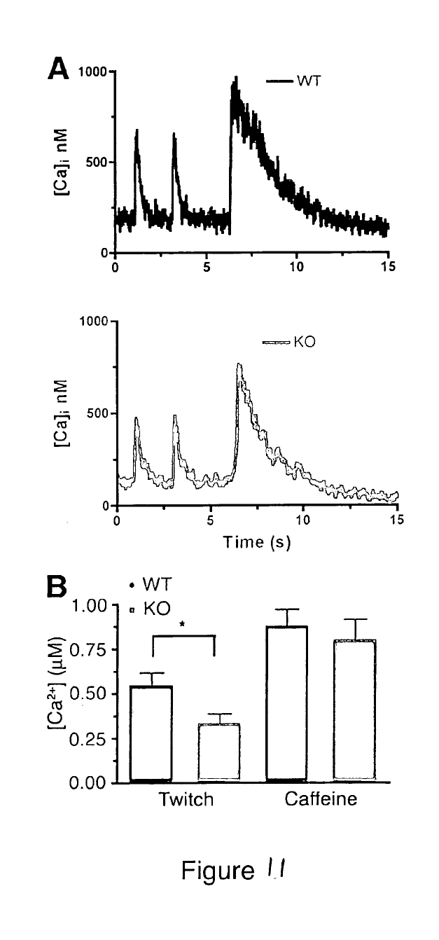

Of note, the decay kinetics of I_{Ca,L} was slower in KO cells compared to WT cells with a decrease in the early fast inactivating component (FIG. 2A). Consistent with previous observations by us and others regarding the role of Akt in cardiac function (Blair et al., 1999; Condorelli et al., 2002; Kim et al., 2003; Sun et al., 2006), both contraction (FIG. 2C) and systolic Ca²⁺ amplitudes (Ca²⁺ transients) (FIG. 2D and FIG. 11A) were significantly depressed (by ˜35% and 30%, respectively, P<0.05) in KO cardiomyocytes compared to WT littermates.

The observed reduction in Ca²⁺ transient amplitude and cardiac contractility could be explained by reduced Ca²⁺ entry into cells via the LTCC, but decreased intracellular Ca²⁺ release from the sarcoplasmic reticulum (SR) may also contribute. However, while the Ca²⁺ transient amplitude between the systolic and diastolic phase (twitch) was smaller in KO cardiomyocytes (FIG. 11B, left bars), no difference in total SR [Ca²⁺ ] content was found (FIG. 11B, right bars), suggesting that the decrease in Ca²⁺ transient amplitude is due only to reduced Ca²⁺ entry. This is consistent with the observed slowing of the early fast inactivation of I_{Ca,L} (FIG. 2A), which is highly dependent on CICR-triggered SR Ca²⁺ release during the action potential (AP) (Richard et al., 2006). Therefore, we conclude that the reduced I_{Ca,L} may contribute to the reduced contractility in KO hearts.

### Akt Regulates the Caᵥα1 Protein Level at the Plasma Membrane

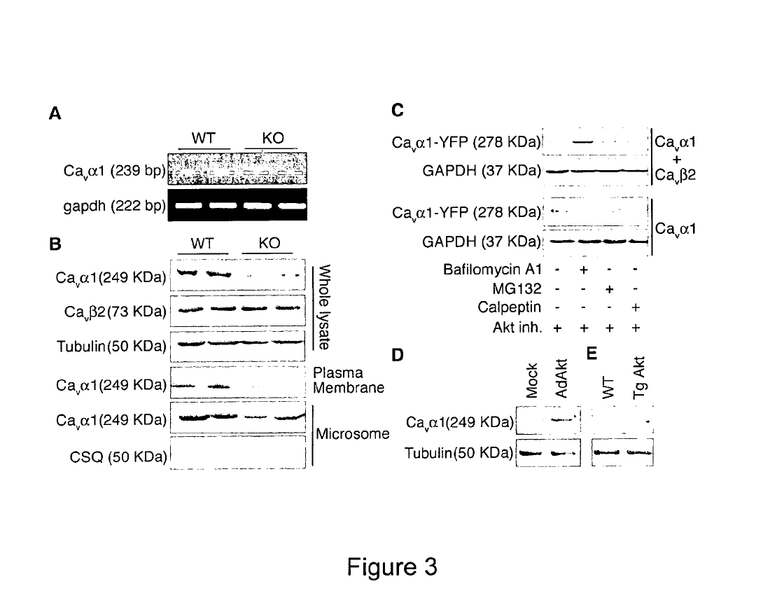

The properties of the Caᵥα1 subunit are known to be markedly affected by LTCC accessory subunits (Bourinet et al., 2004; Catterall, 2000). Among the LTCC accessory subunits expressed in the heart, Caᵥβ2 is known to act as a chaperone for the Caᵥα1 subunit, both as a positive modulator of channel opening probability and for its trafficking from the endoplasmic reticulum (ER) to the plasma membrane (Viard et al., 2004; Yamaguchi et al., 1998). Therefore, supported by previous results (Viard et al., 2004) as well as corroborated by unchanged Caᵥα1 mRNA levels in KO compared to WT hearts (FIG. 3A), we hypothesized that in the heart, an Akt-mediated phosphorylation of the LTCC accessory subunit would mainly affect trafficking of Caᵥα1 protein to the plasma membrane.

However, since the amount of Caᵥα1 was reduced in both microsomal and membrane fractions from KO extracts compared to WT (FIG. 3B), we hypothesized that the reduced Caᵥα1 level observed in KO mice was due to enhanced protein degradation in addition to impaired protein translocation to the plasma membrane. To assess the pathway involved in the Akt-dependent Caᵥα1 protein degradation, three sets of specific cell degradation system inhibitors were examined for their ability to prevent the decrease in Caᵥα1 protein elicited by Akt inhibition. Treatment of Caᵥα1 and Caᵥβ2 cotransfected cells with bafilomycin-A1, an inhibitor of the lysosomal degradation system responsible for the degradation of many membrane proteins (Dice, 1987), prevented the decrease in Caᵥα1 protein induced by Akt inhibition (FIG. 3C, upper panel). Conversely, an ubiquitin/proteasome inhibitor, MG132 failed to protect Caᵥα1 from protein degradation. Similar results were obtained by inhibiting calpain, the intracellular, Ca²⁺-dependent cysteine protease known to be involved in membrane protein degradation (Belles et al., 1988; Romanin et al., 1991). Intriguingly, the bafilomycin-A1-dependent protection effect was abolished in the absence of Caᵥβ2 cotransfection, a condition where Caᵥα1 is retained in the ER (FIG. 3C, lower panel). All together, these results confirm that Akt activity is regulating Caᵥα1 protein density and reveal that in the absence of Akt function, Caᵥα1 is susceptible to lysosome-mediated membrane protein degradation.

Since Caᵥβ2 is the only LTCC accessory subunit containing an Akt-phosphorylation consensus site (Viard et al., 2004), we hypothesized that Caᵥα1 protein degradation at the plasma membrane might result from loss of Caᵥβ2 chaperone activity in the absence of Akt-induced phosphorylation. In support of this hypothesis, forced expression of the active E40K-Akt mutant (AdAkt) restored Caᵥα1 protein levels in isolated cardiomyocytes from KO mice (FIG. 3D). Similarly, cardiomyocytes from transgenic mice expressing constitutively active HA-E40K-Akt (Tg Akt) (Condorelli et al., 2002) showed increased Caᵥα1 levels compared to WT controls (FIG. 3E).

### Akt is Determinant for Caᵥα1 Protein Level Regulation by Direct Phosphorylation of the Caᵥβ2 Chaperone-Subunit

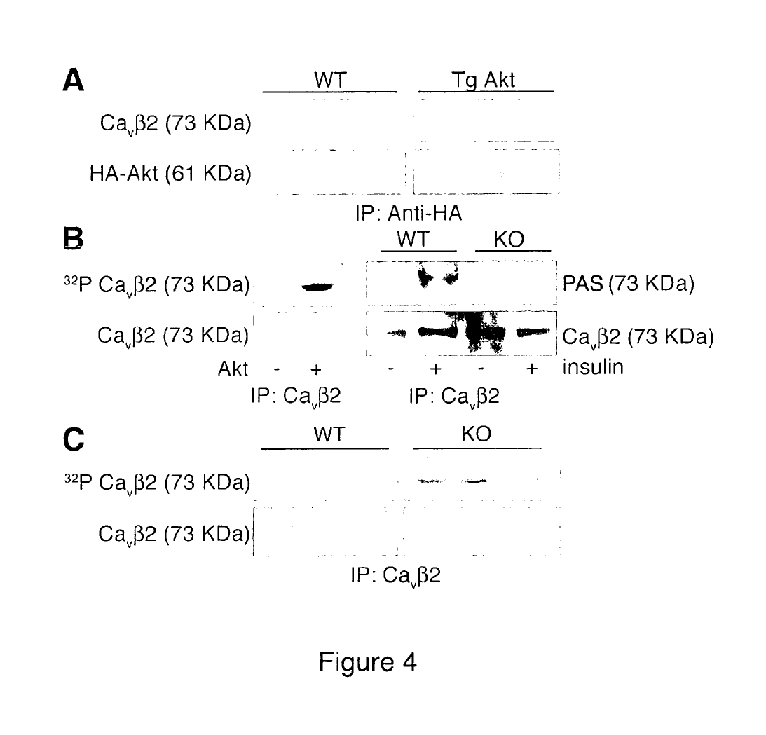

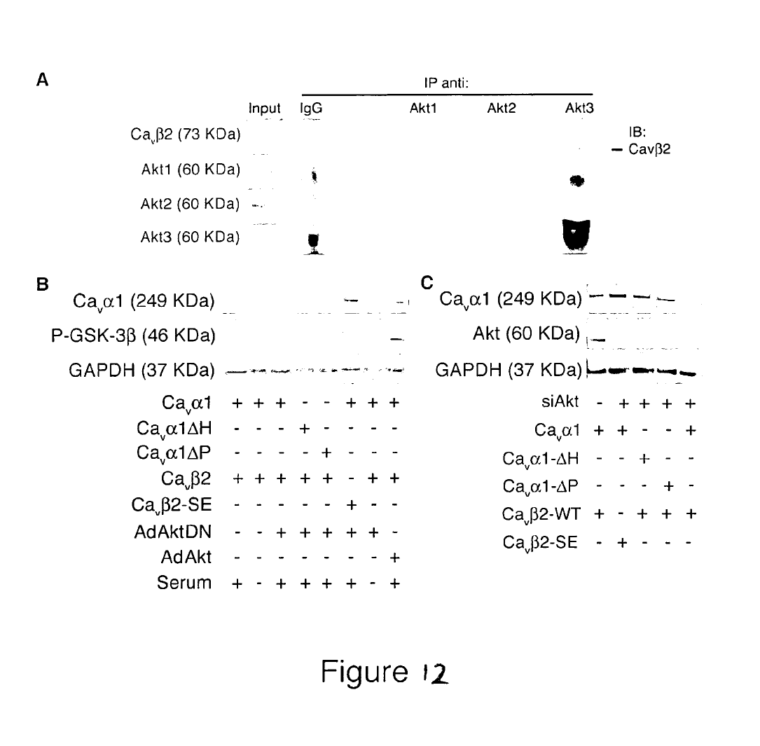

To assess whether Akt is directly involved in modulation of Caᵥβ2 chaperone activity in the heart, we first confirmed the interaction between Akt and Caᵥβ2. Ventricular homogenates derived from either WT or Tg Akt mice were immunoprecipitated with anti-HA antibody and assayed for Caᵥβ2, which revealed association of the Caᵥβ2 subunit with active Akt (FIG. 4A). Similarly, Caᵥβ2 was found to co-immunoprecipitate with insulin-stimulated endogenous Akts (FIG. 12A).

To determine whether Caᵥβ2 can be phosphorylated by Akt, Caᵥβ2-immunoprecipitates from cardiac homogenates were incubated with recombinant active Akt and [γ-³²]ATP. A band corresponding to phosphorylated Caᵥβ2 was detected only in the presence of the kinase (FIG. 4B, left panel). To determine whether the Caᵥβ2 subunit was phosphorylated by Akt in vivo, we treated overnight-starved mice with 1 mU/g insulin to induce activation of Akt (Bayascas et al., 2008). 20 min post treatment, Caᵥβ2 was immunoprecipitated from ventricular homogenates, subjected to Western blot analysis, and probed for phosphorylated Akt consensus sites using PAS (Phospho-Akt Substrate) antibody.

This revealed insulin-stimulated phosphorylation of Caᵥβ2 in WT but not in KO hearts (FIG. 4B, right panel). Furthermore, a back-phosphorylation assay, used to assess the basal state of Caᵥβ2 phosphorylation, revealed a reduction of the basal phosphorylation level of Caᵥβ2 by 36% (p<0.05) in KO mouse ventricle compared to WT (FIG. 4C). Taken together, these data demonstrate that active Akt binds to and phosphorylates Caᵥβ2, the chaperone for Caᵥα1.

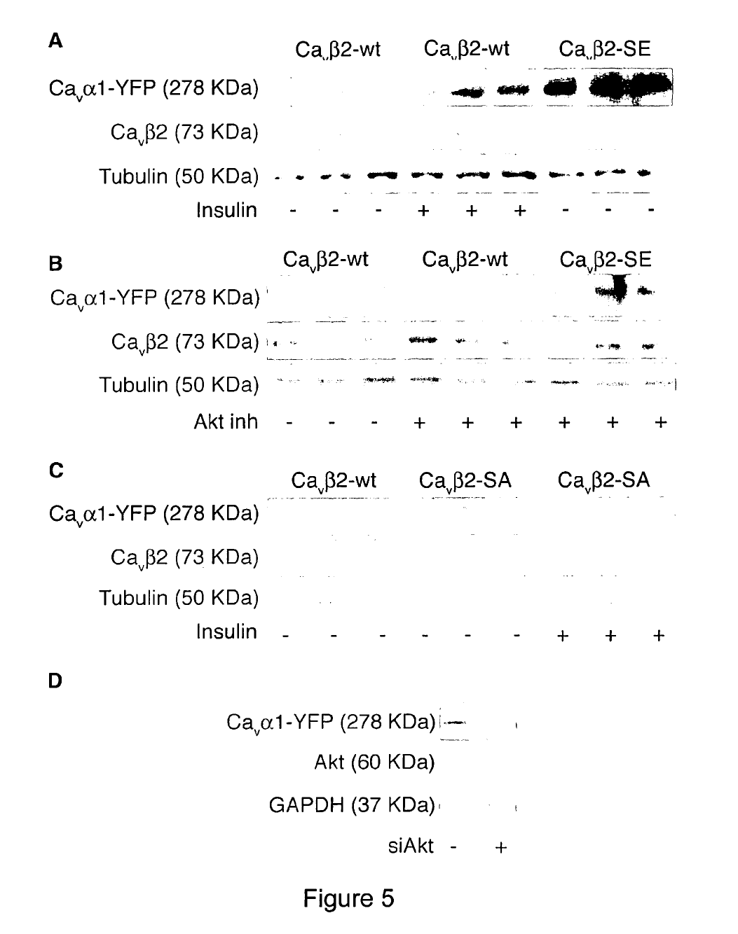

To directly assess whether Akt phosphorylation of Caᵥβ2 protects Caᵥα1 from protein degradation, we constructed a mutant of Caᵥβ2 in which the Serine 625, contained in the putative Akt—consensus site (R—X—X—R—S/T), was replaced by Glutamate (Caᵥβ2-SE) to mimic phosphorylation. Cotransfection of 293T cells with Caᵥα1 and Caᵥβ2-SE resulted in Caᵥα1 protein levels that were increased compared to those found when cotransfected with Caᵥβ2-WT (FIG. 5A). Similarly, Caᵥα1 expression was increased in insulin treated Caᵥβ2-WT cotransfected cells (FIG. 5A). Notably, the active phosphomimic Caᵥβ2-SE also counteracted the downregulation of Caᵥα1 induced by an Akt inhibitor, available from Calbiochem (FIG. 58).

Opposite results were obtained with a dominant-negative Caᵥβ2 mutant in which Serine was replaced by Alanine (Caᵥβ2-SA) to prevent Akt phosphorylation. Indeed, Caᵥα1 protein levels were reduced when coexpressed with Caᵥβ2-SA (FIG. 5C). In addition, insulin stimulation failed to increase Caᵥα1 in the presence of the dominant-negative Caᵥβ2-SA mutant (FIG. 5C). Consistent with the hypothesis that Caᵥα1 protein downregulation relies on Akt kinase activity, overexpression of a dominant negative form of Akt (AdAktDN) resulted in a significant reduction in Caᵥα1 protein levels while forced expression of AdAkt was sufficient to counteract Caᵥα1 reduction in a serum-free condition, where Akt is not phosphorylated (FIG. 12B). Furthermore, suppression of Akt expression in 293T cells by small interfering RNA (siRNA) (siAkt) resulted in reduction of the Caᵥα1 protein level (FIG. 5D).

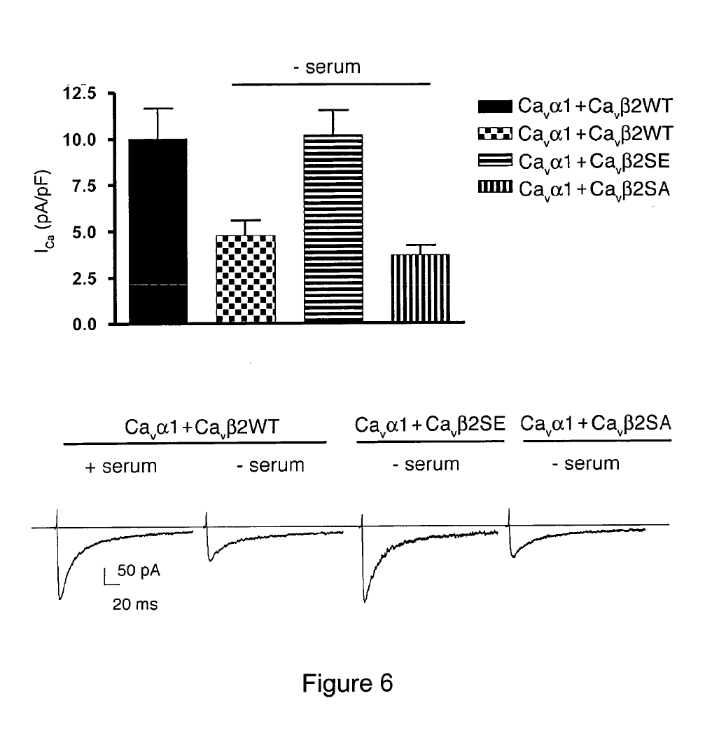

To support the evidence that Akt-dependent phosphorylation of Caᵥβ2 is determinant for Caᵥα1 stability and functionality, we measured the effect of the Caᵥβ2 mutants on Ca²⁺ current. While cotransfection of cells with Caᵥα1 and Caᵥβ2-WT resulted in significant depressed I_{Ca,L} in serum-free medium compared to serum-containing medium where Akt is phosphorylated (data not shown), cotransfection of Caᵥα1 and Caᵥβ2-SE mutant but not Caᵥβ2-SA mutant completely counteracted this reduction (FIG. 6).

### Akt Regulates Caᵥα1 Protein Stability

PEST sequences have been suggested to serve as signals for rapid proteolytic degradation through the cell quality control system (Krappmann et al., 1996; Rechsteiner, 1990; Sandoval et al., 2006; Smith et al., 1993). Notably, PEST-mediated protein degradation has recently been suggested to play an essential role in modulating neuronal Ca²⁺ channel function through regulation of the Caᵥβ3 accessory subunit (Sandoval et al., 2006). Our findings raise the possibility that processing of the Caᵥα1 protein may be affected in a similar way.

To test this hypothesis, we used the web-based algorithm PESTFind (Rogers et al., 1986) in a search for potential Caᵥα1. PEST sequences and found several putative motifs (aa 435-460; 807-820; 847-858; 1732-1745; 1839-1865). Intriguingly, the highest-scored potential PEST sequences obtained are highly conserved among species (Table 1), with one located in the I-II linker of the Caᵥα1 subunit and overlapping with the α1-interacting domain (AID), the primary binding region for Caᵥβ2 (Bodi et al., 2005) (FIG. 7A). To determine whether these PEST sequences are involved in Caᵥα1 degradation control, we generated two in-frame deletion mutants encompassing either the I-II (Caᵥα1-ΔP) or II-III (Caᵥα1-ΔH) cytosolic linker region (FIG. 7A). Western blot and immunofluorescence analyses of serum-starved 293T cells transfected with these mutants revealed higher protein expression levels for both Caᵥα1-ΔP and Caᵥα1-ΔH mutants compared to Caᵥα1-WT, consistent with the hypothesis that these motifs determine Caᵥα1 protein stability (FIG. 7B).

Furthermore, a pulse-chase analysis, with a chase starting 36 h post-cell starvation, revealed markedly increased protein stability of Caᵥα1-ΔP and Caᵥα1-ΔH compared to Caᵥα1-WT (FIG. 7C). In particular, Caᵥα1-WT showed a short half-life typical of proteins containing PEST sequences (Dice, 1987) with a rapid and progressive degradation starting 4 h from the chase and reaching 50% of degradation 25 h after the chase. In contrast, Caᵥα1-ΔP and Caᵥα1-ΔH mutants were less sensitive to degradation and were degraded by only 23% and 15% after 25 h, respectively (P<0.001). Notably, cotransfection of Caᵥβ2-SE with Caᵥα1-WT resulted in a considerable increase in the half-life of Caᵥα1-WT (FIG. 7C).

In addition, transfection of 293T cells with Caᵥα1 PEST sequences fused in-frame with GFP resulted in marked instability of GFP, as shown by both Western blot and immunofluorescence analyses (FIG. 7D), providing further evidence that these motifs are determinants for Caᵥα1 protein stability. Consistent with the hypothesis that Akt-mediated protection of Caᵥα1 degradation acts through PEST sequences, overexpression of AdAktDN or siAkt had no significant effect on protein levels of either Caᵥα1-ΔP or Caᵥα1-ΔH mutants (FIG. 12B-C). To assess whether the observed PEST-mechanism is due to a direct Akt-dependent interaction between Caᵥβ2 and Caᵥα1, we performed in vitro binding assays using in vitro translated ³⁵S-methionine-labeled Caᵥα1 cytosolic domains and GST-fused Caᵥβ1 C-terminal coiled coil region. Notably, direct interaction took place between the Akt-phosphorylated Caᵥβ2 C-terminal coiled coil region and the Caᵥα1 C-terminal domain (FIG. 7E). No interactions were found with other Caᵥα1 cytosolic domains (data not shown), although it cannot be excluded that other binding sites may exist.

To assess whether PEST-deleted Caᵥα1 channels are still functional, traffic appropriately to the membrane, and associate with the Caᵥβ2 subunit, we measured Ca²⁺ current in Caᵥα1-ΔH mutant transfected cells. No significant differences in I_{Ca,L} were found in cells transfected with Caᵥα1-WT compared to Caᵥα1-ΔH (FIG. 7F). Conversely, while serum deprivation resulted in I_{Ca,L} reduction in Caᵥα1-WT transfected cells, no significant changes were observed in Caᵥα1-OH mutant transfected cells (FIG. 7F). This confirms that PEST-deleted Caᵥα1-ΔH is resistant to rapid protein degradation and maintains its integrity and physiological function.

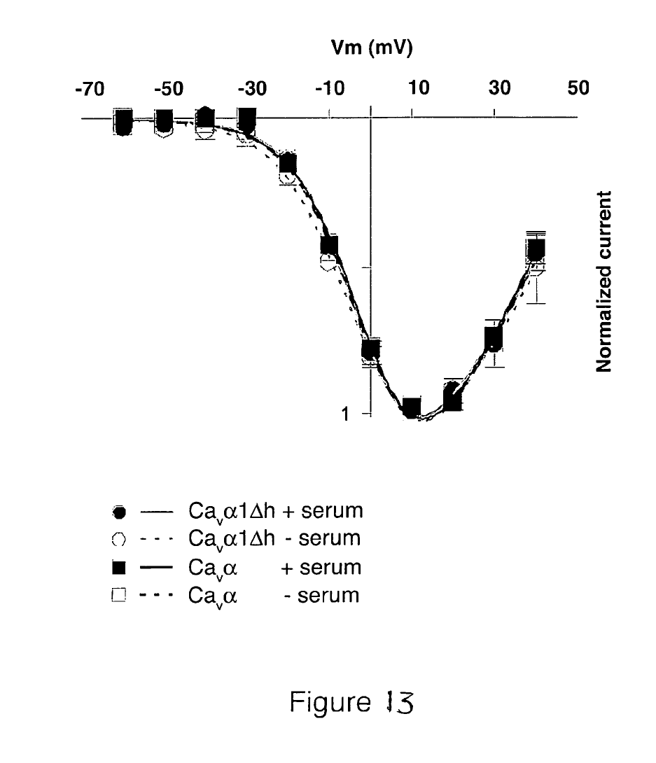

Furthermore, current-voltage analysis (IV curves) revealed that neither serum deprivation, nor PEST-H deletion modify steady-state activation parameters (FIG. 13). Also, all electrophysiological experiments were performed at a holding potential of −80 mV, a value far away from the potential for half steady-state inactivation (V0.5) of I_{Ca,L}, indicating that a change in the macroscopic current properties of Caᵥ1.2 is unlikely.

Taken together, our results suggest that Akt-mediated phosphorylation of Caᵥβ2 regulates Caᵥα1 density through protection of Caᵥα1 PEST motifs from the cell protein degradation machinery. Impairment of this mechanism is expected to result in dysregulation of cardiomyocyte contractile function.

### Materials and Methods

### Generation of Genetically Modified Mice

Cardiac-specific PDK1 inducible knockout mice (MerCreMer-α-MHC PDK1) were generated by breeding PDK1^{floxed/floxed} transgenic mice (Williams et al., 2000), with mice expressing the cardiac-specific MerCreMer-α-MHC promoter-driven Cre recombinase gene (Sohal et al., 2001). The resulting background strain of the MerCreMer mice was C57BL/6-SV129 and was unchanged throughout all experiments. Control animals used in this study were PDK1^{floxed/floxed} littermates, not expressing the Cre recombinase gene, and treated with the same Tamoxifen regiment. Tamoxifen dissolved in corn oil was injected intraperitoneally once a day at a dose of 75 mg/Kg body weight. Male animals, 7-8 weeks old were used. All animal procedures were performed in accordance with the Guide for the Care and Use of Laboratory Animals and approved by the Institutional Animal Care and Use Committee.

### Reverse Transcription-PCR (RT-PCR) Analysis

Sequences of oligonucleotide primers to perform RT-PCR are available from the authors on request.

### Culture and Treatment of Mouse Cardiomyocyte Cells

Isolation of ventricular myocytes was carried out as previously described (Care et al., 2007). Cells were infected with an adenovector expressing either no transgene (mock), HA-E40K-Akt (AdAkt), or Akt-K179M (AdAktDN) at m.o.i. 100 and harvested 48 h post infection. The viral vector was amplified and purified in 3% Sucrose/PBS by ViraQuest, Inc. (North Liberty, Iowa).

Cell Culture and cDNA Mutagenesis

Cell transfection was performed in serum-starved medium using LipoFectamine2000 (Invitrogen) according to the manufacturer's instructions. 5 μM Akt-XI inhibitor (Calbiochem), insulin (Sigma), 1 μM bafilomycin-A 1 (Sigma), 25 μM MG132 (Calbiochem), and 25 μM Calpeptin (Calbiochem) were used as described. Cacnb2 cDNA (complete cds, cDNA clone MGC:129335, IMAGE:40047531, ATCC #10959168) was cloned in the pcDNA3 vector. Site-directed mutagenesis was performed using the QuikChange Site-Directed Mutagenesis Kit (Stratagene, La Jolla, Calif.). Caᵥα1 PEST deletion mutants and GFP fusion proteins were generated by PCR. A lentivirus vector was generated and used as an expression vector for siRNA-mediated silencing of the AKT gene (siAKT). The used sequence (5′-tgcccttctacaaccaggatt-3′) was chosen in a conserved region between rat, mouse, and human and has been validated for targeting Akt 1 and Akt2 (Katome et al., 2003). All constructs were confirmed by DNA sequencing. Primer sequences are available from the authors on request.

### Ca²⁺ Current Measurement

Macroscopic I_{ce}, was recorded at room temperature (˜22° C.) using the whole-cell patch clamp technique in native cells as previously described (Aimond et al., 2005; Maier et al., 2003). External recording solution contained (in mM): 136 TEA-Cl, 2 CaCl₂, 1.8 MgCl₂, 10 HEPES, 5 4-aminopyridine, and 10 glucose (pH 7.4 with TEA-OH). Pipette solution contained (in mM): 125 CsCl, 20 TEA-Cl, 10 EGTA, 10 HEPES, 5 phosphocreatine, 5 Mg₂ATP, and 0.3 GTP (pH 7.2 with CsOH). Myocytes were held at −80 mV and 10 mV depolarizing steps from −50 mV to +50 mV for 300 ms were applied. Analysis was performed using a microscope nikon diaphot 200 (objective lenses nikon cfwn 10×/20). pclamp 9 (axon laboratory) was used as acquisition software. For electrophysiological recordings of recombinant Caᵥα1 currents, tsA-201 cells were transfected in OptiMEM with a DNA mix containing plasmids encoding YFP-Caᵥα1, Caᵥβ2 subunit (either Caᵥβ2-WT, Caᵥβ2-SE, or Caᵥβ2-SA), Caᵥα2δ1 subunit, and CD8 (in a ratio 1:2:0.5:0.1). After 24 h, cells were cultured in DMEM with or without serum for 36 h and electrophysiological recordings were performed on cells expressing both YFP-Caᵥα1 and CD8, which is identified using anti-CD8 coated beads (Dynabeads, Dynal). The extracellular solution contained (in mM): 135 NaCl, 20 TEAC1, 5 CaCl₂, 1 MgCl₂, and 10 HEPES (pH adjusted to 7.4 with KOH, ˜330 mOsM). Borosilicate glass pipettes have a typical resistance of 1.5-3 MW when filled with an internal solution containing (in mM): 140 CsCl, 10 EGTA, 10 HEPES, 3 Mg-ATP, 0.6 GTPNa and 2 CaCl₂ (pH adjusted to 7.2 with KOH, ˜315 mOsM). Analysis was performed using a microscope Olympus x71. Data acquisition with software pclamp9.

Fluorescent Measurement of [Ca²⁺]ᵢ;

Isolated myocytes were loaded with 5 μM Fura-PE3 AM (TefLabs) and analyzed as previously described (Bassani et al., 1994; DeSantiago et al., 2002). Analysis was performed using a Nikon microscope. Data acquisition and analysis were performed using axon Pclamp software (clampex and clampfit v8.2).

### Akt and PKC Kinase Assay

Myocardial tissue lysates were tested using the Akt Kinase Assay Kit (Cell Signaling) and PKC (Upstate Biotechnology) according to the manufacturer's instructions.

### Western Blot Analysis and Antibodies

Proteins expression was evaluated in total lysates or cell fractions by Western blot analysis according to standard procedures. Antibodies against the following proteins were used: Caᵥα1 (Novus Biologicals), Caᵥα1, and Caᵥβ2 (kindly provided by Dr. Hannelore Haase, Max Delbrück Center for Molecular Medicine), Ryr, and Ryr2-P2809 (kindly provided by Dr. Andrew Marks, Columbia University), PDK1 (Calbiochem), Akt1, Akt2, Akt3, Akt, Akt-P308, and anti-phospho-(Ser/Thr)-Akt substrate (PAS) (Cell Signaling Technology), PLN, and PLN-P16 (Novus Biologicals), Calsequestrin (BD Transduction Laboratories), Caspase-3 (Cell Signaling), HA (Roche), GFP/YFP (GeneTex, Inc), tubulin (Novus Biologicals), GSK3r3 (Cell Signaling), and GAPDH (Cell Signaling Technology). ImageJ software (NIH) was used to perform densitometry analyses.

### Tissue Preparation, Immunoprecipitation, and In Vitro Phosphorylation

When described, overnight fasted mice were injected i.p. with insulin (1 mU/g) or saline solution. 20 min after injection, the hearts were rapidly extracted, freeze clamped in liquid nitrogen, and homogenized to a powder in liquid nitrogen. In vitro phosphorylation assays on immunoprecipitates were performed as described elsewhere (Haase et al., 1999).

### Cell Fractionation

Pulverized hearts were homogenized in ice-cold solution 1 (300 mM Sucrose, 10 mM Tris-HCl, pH 7.5, 1 mM EDTA, 1 mM EGTA, 50 mM NaF, 1 mM Na₃VO₄, and protease inhibitors) (1.5 ml/ventricle) by three bursts of 10 s in a Polytron homogenizer. Homogenates were then incubated for 15 min on ice (whole homogenates). Samples were spun at 1000×g for 10 min at 4° C. Pellets were washed in solution 1, spun at 1000×g for 10 min at 4° C., and supernatants were filtered through 4 layers of cheese clothes and centrifuged at 10000×g for 30 mM at 4° C. Supernatants were then centrifuged at 143000×g for 30 mM at 4° C. and pellets were resuspended in solution 3 (600 mM KCl, 30 mM Tris-HCl, pH 7.5, 300 mM Sucrose, 1 mM EDTA, 1 mM EGTA, 50 mM NaF, 1 mM Na₃VO₄, and protease inhibitors). Supernatants were saved as cytosolic fraction. Resuspended pellets from a further centrifugation at 143000×g for 45 mM at 4° C. were resuspended in solution 4 (100 mM KCl, 20 mM Tris-HCl, pH 7.5, 300 mM Sucrose, 1 mM EDTA, 1 mM EGTA, 50 mM NaF, 1 mM Na₃VO₄, and protease inhibitors) and saved as ER fraction. All aliquots were stored at −80° C.

### Histology and Confocal Microscopy

Fixation, staining, and confocal analysis was performed as previously described (Care et al., 2007). Confocal microscopy was performed using a confocal microscope (Radiance 2000; Bio-Rad) with a 60× plan—Apochromat NA 1.4 objective (Carl Zeiss MicroImaging, Inc.). Individual images (1,024×1,024) were converted to tiff format and merged as pseudocolor RGB images using Imaris (Bitplane AG).

### Pulse Chase and Immunoprecipitation Experiments

36 h post transfection, 293T cells were starved for 30 min in methionine- and cysteine-free DMEM medium (Sigma) and were then labeled for 30 mM by adding 500 μCi [35S]-L-methionine and 2 mM L-cysteine. Radioactive media was eventually washed out with PBS (time 0 pulse) and replaced with normal DMEM. Time points were at 4, 10, and 25 h post pulse. Anti-GFP polyclonal IgG (GTX20290) was used for immunoprecipitation. Radioactivity was quantitated with ImageQuant 5.2 software (GE Amersham).

### GST Pull-Down Assay

Affinity-purified GST-fusion proteins were generated using a pGEX system (Amersham) and phosphorylated as described below. GST-fusion protein bound to glutathione-Sepharose 4B beads (Amersham) was incubated with 25 μl of ³⁵S labeled methionine protein with moderate shaking at 25° C. for 2 h in 200 μl of binding buffer containing 20 mM HEPES, pH 7.9, 1 mM EDTA, 10% glycerol, 0.15 M KCl, 0.05% Nonidet P-40, and 1 mM DTT. ³⁵S labeled probes were generated from the C-terminal region of Caᵥα1 cDNA fragments under control of the T7 promoter using the TnT Quick Coupled Reticulocyte Lysate System (L1170, Promega) and washed three times with washing buffer (20 mM HEPES, pH 7.9, 1 mM EDTA, 10% glycerol, 250 m M KCl, 0.1% Nonidet P-40) and centrifuged. Bound proteins were eluted in SDS sample buffer, subjected to SDS-PAGE, and detected by autoradiography. Recombinant GST-Caᵥβ2 beads or GST beads were phosphorylated by incubation with recombinant Akt (Millipore). Briefly, 5 μg of GST-Caᵥβ2 or GST beads was incubated at 30° C. for 45 min in a solution (50 μl) containing 2 μg of activated Akt kinase, 10 mM Hepes-KOH at pH 7.5, 50 mM γ-glycerophosphate, 50 mM NaCl, 1 mM dithiothreitol, 10 mM MnCl₂, and 1 mM ATP.

### Statistical Analysis

Statistical comparison was carried out within at least 3 independent experiments by paired or unpaired Student t-test, while comparison between groups was analyzed by 1-way repeated-measures ANOVA combined with a Newman-Keuls post-test to compare different values using Prism 4.0 software (GraphPad Software, CA). Differences with P<0.05 were considered statistically significant.

TABLE 1
PEST sequences are highly conserved in Caᵥa1.

| SEQ | | | | |
|---|---|---|---|---|
| ID | | | | PestFind |
| NO: | Species | | Sequences | score |
| 5 | Mouse | Pest I | 435 KGYLDWITQAEDIDPENEDEGMDEDK 460 | +8.45 |
| 6 | Rat | Pest I | 476 KGYLDWITQAEDIDPENEDEGMDEDK 501 | +8.45 |
| 7 | Human | Pest I | 446 KGYLDWITQAEDIDPENEDEGMDEEK 471 | +8.66 |
| 8 | Mouse | Pest II | 807 KSITADGESPPTTK 820 | +9.45 |
| 9 | Rat | Pest II | 848 KSITADGESPPTTK 861 | +9.45 |
| 10 | Mouse | Pest | 837 HSNPDTAGEEDEEEPEMPVGPR 858 | +19.51 |
| | | III | | |
| 11 | Rat | Pest | 878 HSNPDTAGEEDEEEPEMPVGPR 899 | +19.51 |
| | | III | | |
| 12 | Human | Pest II | 845 KSPYPNPETT GEEDEEEPEMPVGPR 869 | +20.26 |
| 13 | Mouse | Pest | 1732 KTGNNQADTESPSH 1745 | +5.5 |
| | | IV | | |
| 14 | Rat | Pest | 1772 KTGNNQADTESPSH 1785 | +5.5 |
| | | IV | | |
| 15 | Mouse | Pest | 1839 RMSEEAEYSEPSLLSTDMFSYQEDEH 1865 | +5.86 |
| | | V | | |
| 16 | Human | Pest | 1937 HDTEACSEPSLLSTEMLSYQDDENR 1961 | +7.54 |
| | | IV | | |
| 17 | Human | Pest | 2214 RGAPSEEELQDSR 2226 | +7.71 |
| | | V | | |

Occurrence of PEST sites within the amino acid sequence of Caᵥal from mouse, rat, and human. Amino acid identity is highlighted in underline.

TABLE 2
Echocardiography analysis of WT and KO mice at day
7 following initiation of tamoxifen injections.

| | Basal (n = 10) | KO (n = 10) |
|---|---|---|
| HR (bpm) | 506 ± 5 | 492 ± 9 |
| BW (g) | 21 ± 4 | 21 ± 4 |
| LVIDd/BW | 0.16 ± 0.02 | 0.20 ± 0.03* |
| IVSd | 0.57 ± 0.02 | 0.51 ± 0.02** |
| LVIDd | 3.35 ± 0.33 | 4.24 ± 0.19** |
| LVPWd | 0.59 ± 0.06 | 0.52 ± 0.01 |
| IVSs | 0.96 ± 0.09 | 0.67 ± 0.08** |
| LVIDs | 1.80 ± 0.37 | 3.66 ± 0.28** |
| LVPWs | 1.16 ± 0.09 | 0.84 ± 0.07** |
| % FS | 46.5 ± 7.02 | 13.79 ± 5.39** |
| EDD/PWD | 5.73 ± 0.60 | 8.16 ± 0.25** |
| VCF (circ/s) | 8.85 ± 1.40 | 3.19 ± 1.19** |
| LVM (d)(mg) | 55.06 ± 12.86 | 73.89 ± 7.74* |
| LVPWd/LVIDd | 0.18 ± 0.02 | 0.12 ± 0.02* |
| HW/BW | 0.0054 ± 0.0010 | 0.0067 ± 0.0011* |

Values are expressed as mean ± SD. BW, body weight; HW, heart weight; LVIDd, left ventricular internal end-diastolic diameter; LVIDs, left ventricular internal end-systolic diameter; IVSd/s, interventricular septum thickness in diastole/systole; LVPWd/s, left ventricle posterior wall thickness in diastole/systole; FS, fractional shortening; VCF, velocity of circumferential fiber shortening calculated as FS divided by ejection time multiplied by the square root of the RR interval.
*P < 0.05,
**P < 0.01,
***P < 0.001.

FIG. 9 shows additional biochemical, histological, and echocardiographic analyses of mouse lacking PDK1 expression. FIG. 10 shows (A) SERCA 2 level and (B) phosphorylation of specific PKA regulatory sites in two SR Ca²⁺-regulatory proteins, ryanodine receptor (Ryr2-P2809) and phospholamban (PLN-P16). FIG. 11 shows (A) representative Ca²⁺ traces and (B) twitch Ca²⁺ transient amplitude in KO compared to WT cardiomyocytes. FIG. 12 shows (A) co-immunoprecipitation of Caᵥβ2 with insulin-activated Akt isoforms; effects of (B) dominant active and negative Akt as well as (C) siAkt on the Caᵥα1 protein level. FIG. 13 shows current-voltage analysis (IV curves) of cells transfected with Caᵥα1-WT or Caᵥα1-ΔH in normal or serum-free conditions. Table 2 shows echocardiography analysis values of WT and KO mice.

Abbreviation lists: AdAkt, Ad-HA-E40K-Akt; AdAktDN, Ad-AktK179M; AID, α1-interacting domain; Caᵥα1, pore-forming Ca²⁺ alpha1 channel subunit; Caᵥβ2, Ca²⁺ beta2 channel accessory subunit; CICR, calcium-induced calcium release; I_{Cax}, Ca²⁺ current; IGF-1, insulin-like growth factor-1; KO, MerCreMer α-MHC PDK1 mice; LTCC, L-type Ca²⁺ channel; PAS, Phospho-Akt Substrate; PEST, signals for rapid protein degradation; PI3K, phosphatidyl-inositol 3-kinase; PLN, phospholamban; Ryr, ryanodine receptor; Tg Akt, HA-E40K-Akt; WT, wildtype.

### REFERENCES

All references cited herein are hereby incorporated by reference to the extent that they do not conflict with the present invention.
Aimond, F., S. P. Kwak, K. J. Rhodes, and J. M. Nerbonne. 2005. Accessory Kvbeta 1 subunits differentially modulate the functional expression of voltage-gated K+ channels in mouse ventricular myocytes. Circ Res 96:451-8.
Bassani, J. W., R. A. Bassani, and D. M. Bers. 1994. Relaxation in rabbit and rat cardiac cells: species-dependent differences in cellular mechanisms. J Physiol 476:279-93.
Bayascas, J. R., S. Wullschleger, K. Sakamoto, J. M. Garcia-Martinez, C. Clacher, D. Komander, D. M. van Aalten, K. M. Boini, F. Lang, C. Lipina, L. Logie, C. Sutherland, J. A. Chudek, J. van Diepen, P. J. Voshol, J. M. Lucocq, and D. R. Alessi. 2008. Mutation of PDK1 PH domain inhibits PKB/Akt leading to small size and insulin-resistance. Mol Cell Biol 28:3258-72.
Belles, B., J. Hescheler, W. Trautwein, K. Blomgren, and J. O. Karlsson. 1988. A possible physiological role of the Ca-dependent protease calpain and its inhibitor calpastatin on the Ca current in guinea pig myocytes. Pflugers Arch 412:554-6.
Bers, D. M. 2002. Cardiac excitation-contraction coupling. Nature 415:198-205.
Bers, D. M., and E. Perez-Reyes. 1999. Ca channels in cardiac myocytes: structure and function in Ca influx and intracellular Ca release. Cardiovasc Res 42:339-60.
Blair, L. A., K. K. Bence-Hanulec, S. Mehta, T. Franke, D. Kaplan, and J. Marshall. 1999. Akt-dependent potentiation of L channels by insulin-like growth factor-1 is required for neuronal survival. J Neurosci 19:1940-51.
Bodi, I., G. Mikala, S. E. Koch, S. A. Akhter, and A. Schwartz. 2005. The L-type calcium channel in the heart: the beat goes on. J Clin Invest 115:3306-17.
Bourinet, E., M. E. Mangoni, and J. Nargeot. 2004. Dissecting the functional role of different isoforms of the L-type Ca2+ channel. J Clin Invest 113:1382-4.
Care, A., D. Catalucci, F. Felicetti, D. Bonci, A. Addario, P. Gallo, M. L. Bang, P. Segnalini, Y. Gu, N. D. Dalton, L. Elia, M. V. Latronico, M. Hoydal, C. Autore, M. A. Russo, G. W. Dorn, 2nd, O. Ellingsen, P. Ruiz-Lozano, K. L. Peterson, C. M. Croce, C. Peschle, and G. Condorelli. 2007. MicroRNA-133 controls cardiac hypertrophy. Nat Med 13:613-8.
Catalucci, D., and G. Condorelli. 2006. Effects of Akt on cardiac myocytes: location counts. Circ Res 99:339-41.
Catterall, W. A. 2000. Structure and regulation of voltage-gated Ca2+ channels. Annu Rev Cell Dev Biol 16:521-55.
Ceci, M., J. Ross, Jr., and G. Condorelli. 2004. Molecular determinants of the physiological adaptation to stress in the cardiomyocyte: a focus on AKT. J Mol Cell Cardiol 37:905-12.
Condorelli, G., A. Drusco, G. Stassi, R. Roncarati, G. Iaccarino, M. A. Russo, Y. Gu, C. Chung, M. Latronico, C. Napoli, J. Sadoshima, C. M. Croce, and J. Ross, jr. 2002. Akt induces enhanced myocardial contractility and cell size in vivo in transgenic mice. Proc. Natl. Acad Sci. USA.
De Waard, M., D. R. Witcher, and K. P. Campbell. 1994. Functional properties of the purified N-type Ca2+ channel from rabbit brain. J Biol Chem 269:6716-24.
DeSantiago, J., L. S. Maier, and D. M. Bers. 2002. Frequency-dependent acceleration of relaxation in the heart depends on CaMKII, but not phospholamban. J Mol Cell Cardiol 34:975-84.
Dice, J. F. 1987. Molecular determinants of protein half-lives in eukaryotic cells. Faseb J 1:349-57.
Duerr, R. L., S. Huang, H. R. Miraliakbar, R. Clark, K. R. Chien, and J. Ross, Jr. 1995. Insulin-like growth factor-1 enhances ventricular hypertrophy and function during the onset of experimental cardiac failure. J Clin Invest 95:619-27.
Haase, H., T. Podzuweit, G. Lutsch, A. Hohaus, S. Kostka, C. Lindschau, M. Kott, R. Kraft, and I. Morano. 1999. Signaling from beta-adrenoceptor to L-type calcium channel: identification of a novel cardiac protein kinase A target possessing similarities to AHNAK. Faseb J 13:2161-72.
Kamp, T. J., and J. W. Hell. 2000. Regulation of cardiac L-type calcium channels by protein kinase A and protein kinase C. Circ Res 87:1095-102.
Katome, T., T. Obata, R. Matsushima, N. Masuyama, L. C. Cantley, Y. Gotoh, K. Kishi, H. Shiota, and Y. Ebina. 2003. Use of RNA interference-mediated gene silencing and adenoviral overexpression to elucidate the roles of AKT/protein kinase B isoforms in insulin actions. J Biol Chem 278:28312-23.
Kim, Y. K., S. J. Kim, A. Yatani, Y. Huang, G. Castelli, D. E. Vatner, J. Liu, Q. Zhang, G. Diaz, R. Zieba, J. Thaisz, A. Drusco, C. Croce, J. Sadoshima, G. Condorelli, and S. F. Vatner. 2003. Mechanism of enhanced cardiac function in mice with hypertrophy induced by overexpressed Akt. J Biol Chem 278:47622-8.
Kobrinsky, E., S. Tiwari, V. A. Maltsev, J. B. Harry, E. Lakatta, D. R. Abernethy, and N. M. Soldatov. 2005. Differential role of the alpha1C subunit tails in regulation of the Cav1.2 channel by membrane potential, beta subunits, and Ca2+ ions. J Biol Chem 280:12474-85.
Krappmann, D., F. G. Wulczyn, and C. Scheidereit. 1996. Different mechanisms control signal-induced degradation and basal turnover of the NF-kappaB inhibitor IkappaB alpha in vivo. Embo J 15:6716-26.
Lao, Q. Z., E. Kobrinsky, J. B. Harry, A. Ravindran, and N. M. Soldatov. 2008. New Determinant for the CaVbeta2 subunit modulation of the CaV1.2 calcium channel. J Biol Chem 283:15577-88.
Lawlor, M. A., A. Mora, P. R. Ashby, M. R. Williams, V. Murray-Tait, L. Malone, A. R. Prescott, J. M. Lucocq, and D. R. Alessi. 2002. Essential role of PDK1 in regulating cell size and development in mice. Embo J21:3728-38.
Maier, L. S., T. Zhang, L. Chen, J. DeSantiago, J. H. Brown, and D. M. Bers. 2003. Transgenic CaMKIIdeltaC overexpression uniquely alters cardiac myocyte Ca2+ handling: reduced SR Ca2+ load and activated SR Ca2+ release. Circ Res 92:904-11.
McMullen, J. R., T. Shioi, W. Y. Huang, L. Zhang, O. Tamayski, E. Bisping, M. Schinke, S. Kong, M. C. Sherwood, J. Brown, L. Riggi, P. M. Kang, and S. Izumo. 2004. The insulin-like growth factor 1 receptor induces physiological heart growth via the phosphoinositide 3-kinase(p110alpha) pathway. J Biol Chem 279:4782-93.
McMullen, J. R., T. Shioi, L. Zhang, O. Tarnayski, M. C. Sherwood, P. M. Kang, and S.
Izumo. 2003. Phosphoinositide 3-kinase(p110alpha) plays a critical role for the induction of physiological, but not pathological, cardiac hypertrophy. Proc Natl Acad Sci USA 100:12355-60.
Mora, A., A. M. Davies, L. Bertrand, I. Sharif, G. R. Budas, S. Jovanovic, V. Mouton, C. R. Kahn, J. M. Lucocq, G. A. Gray, A. Jovanovic, and D. R. Alessi. 2003. Deficiency of PDK1 in cardiac muscle results in heart failure and increased sensitivity to hypoxia. Embo J 22:4666-76.
Mora, A., D. Komander, D. M. van Aalten, and D. R. Alessi. 2004. PDK1, the master regulator of AGC kinase signal transduction. Semin Cell Dev Biol 15:161-70.
Mukherjee, R., and F. G. Spinale. 1998. L-type calcium channel abundance and function with cardiac hypertrophy and failure: a review. J Mol Cell Cardiol 30:1899-916.
Pereira, L., J. Matthes, I. Schuster, H. H. Valdivia, S. Herzig, S. Richard, and A. M. Gomez. 2006. Mechanisms of [Ca2+]i Transient Decrease in Cardiomyopathy of db/db Type 2 Diabetic Mice. Diabetes 55:608-15.
Quignard, J. F., M. C. Harricane, C. Menard, P. Lory, J. Nargeot, L. Capron, D. Mornet, and S. Richard. 2001. Transient down-regulation of L-type Ca(2+) channel and dystrophin expression after balloon injury in rat aortic cells. Cardiovasc Res 49:177-88.
Rechsteiner, M. 1990. PEST sequences are signals for rapid intracellular proteolysis. Semin Cell Biol 1:433-40.
Richard, S., E. Perrier, J. Fauconnier, R. Perrier, L. Pereira, A. M. Gomez, and J. P. Benitah. 2006. ‘Ca(2+)-induced Ca(2+) entry’ or how the L-type Ca(2+) channel remodels its own signalling pathway in cardiac cells. Prog Biophys Mol Biol 90:118-35.
Rogers, S., R. Wells, and M. Rechsteiner. 1986. Amino acid sequences common to rapidly degraded proteins: the PEST hypothesis. Science 234:364-8.
Romanin, C., P. Grosswagen, and H. Schindler. 1991. Calpastatin and nucleotides stabilize cardiac calcium channel activity in excised patches. Pflugers Arch 418:86-92.
Rota, M., A. Boni, K. Urbanek, E. Padin-Iruegas, T. J. Kajstura, G. Fiore, H. Kubo, E. H. Sonnenblick, E. Musso, S. R. Houser, A. Leri, M. A. Sussman, and P. Anversa. 2005. Nuclear Targeting of Akt Enhances Ventricular Function and Myocyte Contractility. Circ Res 97:1332-41.
Sandoval, A., N. Oviedo, A. Tadmouri, T. Avila, M. De Waard, and R. Felix. 2006. Two PEST-like motifs regulate Ca2+/calpain-mediated cleavage of the CaVbeta3 subunit and provide important determinants for neuronal Ca2+ channel activity. Eur J Neurosci 23:2311-20.
Smith, L. K., M. Bradshaw, D. E. Croall, and C. W. Garner. 1993. The insulin receptor substrate (IRS-1) is a PEST protein that is susceptible to calpain degradation in vitro. Biochem Biophys Res Commun 196:767-72.
Sohal, D. S., M. Nghiem, M. A. Crackower, S. A. Witt, T. R. Kimball, K. M. Tymitz, J. M. Penninger, and J. D. Molkentin. 2001. Temporally regulated and tissue-specific gene manipulations in the adult and embryonic heart using a tamoxifen-inducible Cre protein. Circ Res 89:20-5.
Sun, H., B. G. Kerfant, D. Zhao, M. G. Trivieri, G. Y. Oudit, J. M. Penninger, and P. H. Backx. 2006. Insulin-like growth factor-1 and PTEN deletion enhance cardiac L-type Ca2+ currents via increased PI3Kalpha/PKB signaling. Circ Res 98:1390-7.
Viard, P., A. J. Butcher, G. Halet, A. Davies, B. Nurnberg, F. Heblich, and A. C. Dolphin. 2004. PI3K promotes voltage-dependent calcium channel trafficking to the plasma membrane. Nat Neurosci 7:939-46.
Williams, M. R., J. S. Arthur, A. Balendran, J. van der Kaay, V. Poli, P. Cohen, and D. R. Alessi. 2000. The role of 3-phosphoinositide-dependent protein kinase 1 in activating AGC kinases defined in embryonic stem cells. Curr Biol 10:439-48.
Yamaguchi, H., M. Hara, M. Strobeck, K. Fukasawa, A. Schwartz, and G. Varadi. 1998. Multiple modulation pathways of calcium channel activity by a beta subunit. Direct evidence of beta subunit participation in membrane trafficking of the alpha1C subunit. J Biol Chem 273:19348-56.
Catalucci et al (J. Cell Biol., Vol 184, 23 Mar. 2009, pp 923-933) is a post-published, per-reviewed paper by the present inventors validating the work behind the present invention.

1-23. (canceled)

24. A method of treatment or prophylaxis, comprising administering to a subject a Caᵥβ2 peptide or variant thereof, or a polynucleotide encoding the Caᵥβ2 peptide or variant thereof, wherein the subject is a subject in need of modulation of cardiac inotropism.

25. The method of claim 24, wherein the Caᵥβ2 peptide or variant thereof comprises an Akt consensus sequence, the consensus sequence comprising the sequence set forth as SEQ ID NO: 23 (N′-RTDRS-C′).

26. The method of claim 24, wherein the Caᵥβ2 peptide or variant thereof comprises a “coiled-coil” region, the region comprising the sequence set forth as SEQ ID NO: 3 or 4.

27. The method of claim 24, wherein the Caᵥβ2 peptide or variant thereof comprises the full length Caᵥβ2 peptide sequence set forth as SEQ ID NO: 1 or SEQ ID NO: 2.

28. The method of claim 24, wherein the Caᵥβ2 peptide or variant thereof is a functional mimetic of the Caᵥβ2 peptide capable of modulation of cardiac inotropism.

29. The method of claim 28, wherein the mimetic mimics a phosphorylation of Caᵥβ2.

30. The method of claim 28, wherein the mimetic mimics Caᵥβ2 in its un-phosphorylated state.

31. The method of claim 28, wherein the mimetic is a synthetic molecule that mimics the effect of phosphorylated Caᵥβ2 or un-phosphorylated Caᵥβ2.

32. The method of claim 24, wherein the Caᵥβ2 peptide or variant thereof is capable of preventing proteolytic degradation of Caᵥα1.

33. The method of claim 32, wherein the Caᵥβ2 peptide or variant is capable of preventing proteolytic degradation of a PEST sequence in Caᵥα1.

34. The method of claim 24, wherein the Caᵥβ2 peptide or variant thereof is a variant containing a mutation of a phosphorylation site.

35. The method of claim 34, wherein the phosphorylation site is a Serine or Threonine residue.

36. The method of claim 35, wherein the variant contains the sequence set forth as SEQ ID NO: 1 or 2, with a mutation selected from S625A, S625E, and S625D.

37. The method of claim 24, wherein the Caᵥβ2 peptide, variant, or polynucleotide is a peptide or variant thereof, which is conjugated or coupled to a protein, antibody, or non-peptide synthetic molecule capable of directing the peptide or variant to a specific cell or tissue.

38. The method of claim 24, wherein the Caᵥβ2 peptide, variant, or polynucleotide comprises a polynucleotide, which polynucleotide comprises DNA, RNA, or a mixture thereof, and encodes the Caᵥβ2 peptide or variant, optionally under the control of a suitable promoter.

39. The method of claim 38, wherein the polynucleotide comprises or encodes an antisense polynucleotide, an RNAi polynucleotide, an siRNA polynucleotide, or a microRNA polynucleotide.

40. The method of claim 24, wherein the subject has dilated cardiomyopathy or cardiac hypertrophy and failure, which is primitive or has occurred after myocardial infarction.

41. A protein or polynucleotide, comprising a PEST-binding factor, which is capable of binding to a PEST sequence of a Caᵥα1 polypeptide and preventing the degradation of the Caᵥα1 polypeptide.

42. A polypeptide, comprising a variant of a Caᵥα1 polypeptide, in which either the I-II (Caᵥα1-ΔP) or II-III (Caᵥα1-ΔH) cytosolic linker region of the Caᵥα1 polypeptide has been mutated.

43. The polypeptide of claim 42, wherein the polypeptide does not contain the P sequence set forth as SEQ ID NO: 18, does not contain the P sequence set forth as SEQ ID NO: 19, or does not contain the H sequence set forth as SEQ ID NO: 20.
